SEQUENCE LISTING (31 sequences)

SEQ ID NO: 1 - protein, 288 aa, Homo sapiens
  misc_feature (1)..(288): Human PD-1 amino acid sequence
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 288 aa, Mus musculus
  misc_feature (1)..(288): Mouse PD-1
MWVRQVPWSFTWAVLQLSWQSGWLLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGMVIGIMSALVGIPVLLLLAWALAVFCSTSMSEARGAGSKDDTLKEEPSAAPVPSVAYEELDFQGREKTPELPTACVHTEYATIVFTEGLGASAMGRRGSADGLQGPRPPRHEDGHCSWPL

SEQ ID NO: 3 - protein, 288 aa, Macaca fascicularis; identical to SEQ ID NO: 1
  misc_feature (1)..(288): Cynomolgus Monkey PD-1

SEQ ID NO: 4 - protein, 268 aa, Homo sapiens
  misc_feature (1)..(268): Mature human PD-1 amino acid sequence (without signal sequence)
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 5 - protein, 268 aa, Mus musculus
  misc_feature (1)..(268): Mature mouse PD-1 amino acid sequence (without signal sequence)
SGWLLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGMVIGIMSALVGIPVLLLLAWALAVFCSTSMSEARGAGSKDDTLKEEPSAAPVPSVAYEELDFQGREKTPELPTACVHTEYATIVFTEGLGASAMGRRGSADGLQGPRPPRHEDGHCSWPL

SEQ ID NO: 6 - protein, 268 aa, Macaca fascicularis; identical to SEQ ID NO: 4
  misc_feature (1)..(268): Mature cynomolgus monkey PD-1 (without signal sequence)

SEQ ID NO: 7-19 - skipped (13 entries)

SEQ ID NO: 20 - protein, 118 aa, Artificial sequence
  Synthetic: JTX-4014 VH Sequence
QVQLVQSGAEVKKPGASVKVSCKASGYTFPSYYMHWVRQAPGQGLEWMGIINPEGGSTAYAQKFQGRVTMTRDTSTSTVYMELSSLRSEDTAVYYCARGGTYYDYTYWGQGTLVTVSS

SEQ ID NO: 21 - protein, 9 aa, Artificial sequence
  Synthetic: JTX-4014 HCDR1
YTFPSYYMH

SEQ ID NO: 22 - protein, 17 aa, Artificial sequence
  Synthetic: JTX-4014 HCDR2
IINPEGGSTAYAQKFQG

SEQ ID NO: 23 - protein, 11 aa, Artificial sequence
  Synthetic: JTX-4014 HCDR3
ARGGTYYDYTY

SEQ ID NO: 24 - protein, 107 aa, Artificial sequence
  Synthetic: JTX-4014 VL Sequence
DIQMTQSPSTLSASVGDRVTITCRASQSISSWLAWYQQKPGKAPKLLIYEASSLESGVPSRFSGSGSGTEFTLTISSLQPDDFATYYCQQYNSFPPTFGGGTKVEIK

SEQ ID NO: 25 - protein, 11 aa, Artificial sequence
  Synthetic: JTX-4014 LCDR1
RASQSISSWLA

SEQ ID NO: 26 - protein, 7 aa, Artificial sequence
  Synthetic: JTX-4014 LCDR2
EASSLES

SEQ ID NO: 27 - protein, 9 aa, Artificial sequence
  Synthetic: JTX-4014 LCDR3
QQYNSFPPT

SEQ ID NO: 28 - protein, 444 aa, Artificial sequence
  Synthetic: JTX-4014 heavy chain
QVQLVQSGAEVKKPGASVKVSCKASGYTFPSYYMHWVRQAPGQGLEWMGIINPEGGSTAYAQKFQGRVTMTRDTSTSTVYMELSSLRSEDTAVYYCARGGTYYDYTYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 29 - protein, 214 aa, Artificial sequence
  Synthetic: JTX-4014 light chain
DIQMTQSPSTLSASVGDRVTITCRASQSISSWLAWYQQKPGKAPKLLIYEASSLESGVPSRFSGSGSGTEFTLTISSLQPDDFATYYCQQYNSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 446 aa, Artificial sequence
  Synthetic: vopratelimab heavy chain
EVQLVESGGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 218 aa, Artificial sequence
  Synthetic: vopretelimab light chain
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0378909 A1
(43) Pub. Date: Dec. 1, 2022

(54) Methods of Treating Cancer with Anti-PD-1 Antibodies

(71) Applicant: Jounce Therapeutics, Inc., Cambridge, MA (US)

(72) Inventor: Jian Xu, Cambridge, MA (US)

(73) Assignee: Jounce Therapeutics, Inc., Cambridge, MA (US)

(21) Appl. No.: 17/772,631

(22) PCT Filed: Nov. 4, 2020
(86) PCT No.: PCT/US2020/058808

§ 371 Date: Apr. 28, 2022

Related U.S. Application Data
(60) Provisional application No. 62/930,955, filed on Nov. 5, 2019.

(51) Int. Cl. A61K 39/395; C07K 16/28; A61K 9/00

(52) U.S. Cl. CPC A61K 39/3955; C07K 16/2818; A61K 9/0019; A61K 2039/545

(57) ABSTRACT

Methods of treating cancer with anti-PD-1 antibodies are provided.

### CROSS-REFERENCE TO RELATED APPLICATIONS

This application claims the benefit of priority of U.S. Provisional Application No. 62/930,955, filed Nov. 5, 2019, which is incorporated by reference herein in its entirety for any purpose.

### FIELD OF THE INVENTION

Methods of treating cancer with particular doses of anti-PD-1 antibody are provided.

### BACKGROUND

The Programmed Death 1 (PD-1) protein is an inhibitory member of the CD28 family of receptors, which also includes CD28, CTLA-4, ICOS and BTLA. PD-1 is expressed on the surface of activated B cells, T cells, and myeloid cells. PD-1 contains a membrane proximal immunoreceptor tyrosine inhibitory motif (ITIM) and a membrane distal tyrosine-based switch motif (ITSM). Although structurally similar to CTLA-4, PD-1 lacks the MYPPY motif that is critical for B7-1 and B7-2 binding. In addition, although CD28, ICOS and CTLA-4 (other members of the CD28 family) all have an unpaired cysteine residue allowing for homodimerization, PD-1 is believed to exist as a monomer, lacking the unpaired cysteine residue characteristic in other CD28 family members. The PD-1 receptor has two ligands, PD-ligand-1 (PD-L1) and PD-ligand-2 (PD-L2). The term “PD-L1” refers to the ligand of the PD-1 receptor also known as CD274 and B7H 1. PD-L1 is a 290 amino acid protein with an extracellular IgV-like domain, an extracellular IgC-like domain, a transmembrane domain and a highly conserved intracellular domain of approximately 30 amino acids. PD-L1 is constitutively expressed on many cells such as antigen presenting cells (e.g., dendritic cells, macrophages, and B-cells) and on hematopoietic and non-hematopoietic cells (e.g., vascular endothelial cells, pancreatic islets, and sites of immune privilege). The term “PD-L2” refers to the ligand of the PD-1 receptor also known as CD273 and B7-DC. PD-L2 has an extracellular IgV-like domain, an extracellular IgC-like domain, a transmembrane domain and an intracellular domain of approximately 30 amino acids in humans. PD-L2 has a more restricted expression than PD-L1, with its expression largely confined to hematopoietic cells including macrophages, dendritic cells, some B cell subsets and bone marrow-derived mast cells.

PD-1 functions as an immune checkpoint and works to prevent the activation of T-cells. PD-1 antagonists activate the immune system to attack tumors and have shown success in treating cancers, and in some instances, with less toxicity than other chemotherapeutic treatments. PD-1 antagonists can also be used in combination regimens with other chemotherapeutic agents. Currently approved PD-1 antagonists include anti-PD-1 antibodies, Opdivo® and Keytruda®, and anti-PD-L1 antibody, Tecentriq™.

### SUMMARY

Provided herein are methods of treating cancer by administering an anti-PD-1 antibody periodically. In some embodiments, methods of treating cancer in a subject (e.g., a human patient) in need thereof are provided comprising administering multiple doses of an anti-PD-1 antibody to the subject.

In some embodiments, methods of treating cancer in a subject are provided, comprising administering a dose of an anti-PD-1 antibody to said subject either at 1000 mg/kg once every 6 weeks or at 400-600 mg/kg once every 3 weeks. In some embodiments, the anti-PD-1 antibody comprises a heavy chain complementarity determining region 1 (HCDR1) comprising the amino acid sequence of SEQ ID NO: 21, a HCDR2 comprising the amino acid sequence of SEQ ID NO: 22, a HCDR3 comprising the amino acid sequence of SEQ ID NO: 23, a light chain CDR1 (LCDR1) comprising the amino acid sequence of SEQ ID NO: 25, a LCDR2 comprising the amino acid sequence of SEQ ID NO: 26, and a LCDR3 comprising the amino acid sequence of SEQ ID NO: 27.

In some embodiments, the anti-PD-1 antibody comprises a heavy chain variable region comprising the amino acid sequence of SEQ ID NO: 20 and a light chain variable region comprising the amino acid sequence of SEQ ID NO: 24. In some embodiments, the anti-PD-1 antibody comprises a heavy chain comprising the amino acid sequence of SEQ ID NO: 28 and a light chain comprising the amino acid sequence of SEQ ID NO: 29.

In some embodiments, the dose is administered every 6 weeks at 1000 mg/kg. In some embodiments, the dose is administered every 3 weeks at 400-600 mg/kg. In some embodiments, the dose is administered every 3 weeks at 400 mg/kg.

In some embodiments, the dose is administered every 3 weeks at 500 mg/kg. In some embodiments, the dose is administered every 3 weeks at 600 mg/kg.

In some embodiments, the subject is administered the anti-PD-1 antibody for a period of about 12 weeks. In some embodiments, the subject is administered the anti-PD-1 antibody for a period of about 18 weeks. In some embodiments, the subject is administered the anti-PD-1 antibody for a period of about 24 weeks. In some embodiments, the subject is administered the anti-PD-1 antibody for a period of about 52 weeks.

In some embodiments, the subject has a cancer selected from melanoma, non-small cell lung cancer (NSCLC), renal cell carcinoma (RCC) (e.g., clear cell RCC), gastric cancer, bladder cancer, endometrial cancer, MSI-H cancer of any organ, diffuse large B-cell lymphoma (DLBCL), Hodgkin's lymphoma, ovarian cancer (e.g., endometrioid ovarian cancer), head & neck squamous cell cancer (HNSCC), acute myeloid leukemia (AML), rectal cancer, refractory testicular cancer, small cell lung cancer (SCLC), small bowel cancer, metastatic cutaneous squamous cell cancer, cervical cancer, MSI-high colon cancer, esophageal cancer, mesothelioma, breast cancer, and triple negative breast cancer (TNBC). In some embodiments, the cancer is selected from melanoma, gastric cancer, head & neck squamous cell cancer (HNSCC), non-small cell lung cancer (NSCLC), and triple negative breast cancer (TNBC).

In some embodiments, the method comprises administering an anti-PD-1 antibody and at least one additional therapeutic agent. In some embodiments, the additional therapeutic agent is administered concurrently or sequentially with the anti-PD-1 antibody. In some embodiments, the additional therapeutic agent is an anti-ICOS antibody. In some embodiments, the anti-ICOS antibody is GSK3359609, BMS-986226, or KY1044. In some embodiments, the anti-ICOS antibody is vopratelimab.

In some embodiments, each dose of the anti-ICOS agonist antibody is 0.1 mg/kg. In some embodiments, each dose of the anti-ICOS agonist antibody is 0.03 mg/kg.

In some embodiments, the subject is a human.

### BRIEF DESCRIPTION OF THE DRAWINGS

FIG. 1 shows changes of an anti-PD-1 antibody (JTX-4014) concentration over time after administration of JTX-2014 at 80 mg, 240 mg, 400 mg, 500 mg, 800 mg, 1000 mg, and 1200 mg, respectively at a three-week interval.

FIG. 2 shows changes of an anti-PD-1 antibody (JTX-4014) concentration over time after administration of JTX-2014 at 800 mg, 1000 mg, and 1200 mg, respectively at a six-week interval.

FIG. 3 shows simulated serum concentration over time for Q6W dosing at 800, 1000, and 1200 mg/kg for 3 doses for a period of 18 weeks.

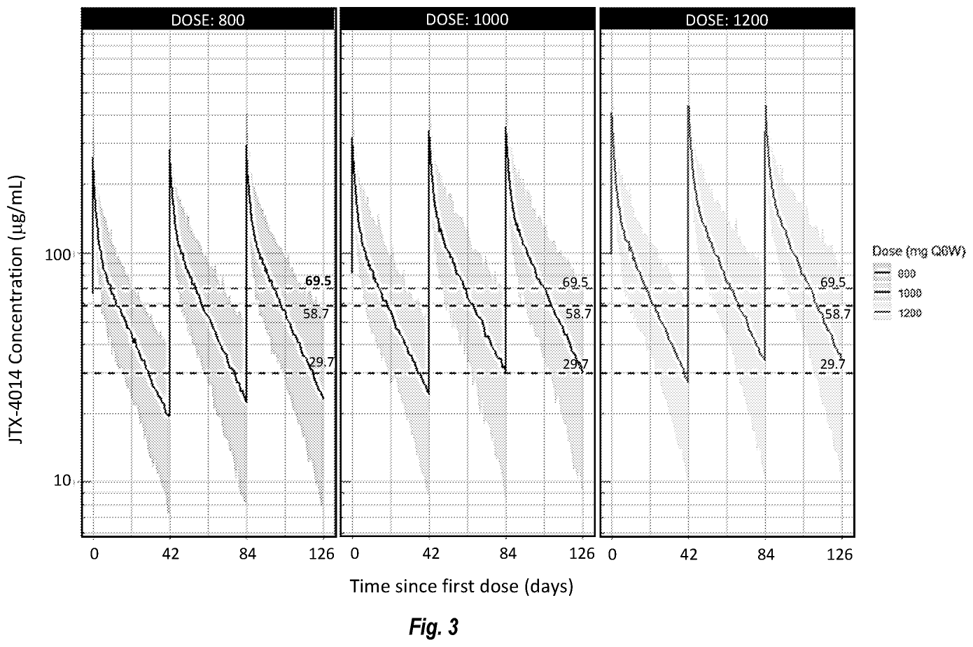

### DETAILED DESCRIPTION OF SOME EMBODIMENTS

In general, methods of treatment using antibodies to Programmed Death 1 (PD-1) are provided. Such methods include, but are not limited to, methods of treating cancer. In some embodiments, methods of treating cancer comprise administering an anti-PD-1 antibody (e.g., JTX-4014, described below) periodically. In some embodiments, a dose of the anti-PD-1 antibody is administered once every 3 weeks. In some embodiments, a dose of the anti-PD-1 antibody is administered once every 6 weeks. In some embodiments, a dose of the anti-PD-1 antibody at 400-600 mg/kg or 500 mg/kg is administered once every 3 weeks. In some embodiments, a dose of the anti-PD-1 antibody at 1000 mg/kg is administered once every 6 weeks.

The section headings used herein are for organizational purposes only and are not to be construed as limiting the subject matter described.

All references cited herein, including patent applications, patent publications, and Genbank Accession numbers are herein incorporated by reference, as if each individual reference were specifically and individually indicated to be incorporated by reference in its entirety.

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993); and updated versions thereof.

### I. Definitions

Unless otherwise defined, scientific and technical terms used in connection with the present disclosure shall have the meanings that are commonly understood by those of ordinary skill in the art. Further, unless otherwise required by context or expressly indicated, singular terms shall include pluralities and plural terms shall include the singular. For any conflict in definitions between various sources or references, the definition provided herein will control.

It is understood that embodiments of the invention described herein include “consisting” and/or “consisting essentially of” embodiments. As used herein, the singular form “a”, “an”, and “the” includes plural references unless indicated otherwise. Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

As is understood by one skilled in the art, reference to “about” a value or parameter herein includes (and describes) embodiments that are directed to that value or parameter per se. For example, description referring to “about X” includes description of “X”.

“PD-1” and “programmed death 1” as used herein refer to any native PD-1 that results from expression and processing of PD-1 in a cell. The term includes PD-1 from any vertebrate source, including mammals such as primates (e.g., humans and cynomolgus monkeys) and rodents (e.g., mice and rats), unless otherwise indicated. The term also includes naturally occurring variants of PD-1, e.g., splice variants or allelic variants. The amino acid sequence of an exemplary human PD-1 precursor protein (with signal sequence, amino acids 1-20) is shown in SEQ ID NO: 1. The amino acid sequence of an exemplary mature human PD-1 is shown in SEQ ID NO: 4. The amino acid sequence of an exemplary mouse PD-1 precursor protein (with signal sequence, amino acids 1-20) is shown in SEQ ID NO: 2. The amino acid sequence of an exemplary mature mouse PD-1 is shown in SEQ ID NO: 5. The amino acid sequence of an exemplary cynomolgus monkey PD-1 precursor protein (with signal sequence, amino acids 1-20) is shown in SEQ ID NO: 3. The amino acid sequence of an exemplary mature cynomolgus monkey PD-1 is shown in SEQ ID NO: 6.

The term “specifically binds” to an antigen or epitope is a term that is well understood in the art, and methods to determine such specific binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to an PD-1 epitope is an antibody that binds this epitope with greater affinity, avidity, more readily, and/or with greater duration than it binds to other PD-1 epitopes or non-PD-1 epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specificity” refers to the ability of a binding protein to selectively bind an antigen.

As used herein, the term “epitope” refers to a site on a target molecule (for example, an antigen, such as a protein, nucleic acid, carbohydrate or lipid) to which an antigen-binding molecule (for example, an antibody, antibody fragment, or scaffold protein containing antibody binding regions) binds. Epitopes often include a chemically active surface grouping of molecules such as amino acids, polypeptides or sugar side chains and have specific three-dimensional structural characteristics as well as specific charge characteristics. Epitopes can be formed both from contiguous and/or juxtaposed noncontiguous residues (for example, amino acids, nucleotides, sugars, lipid moiety) of the target molecule. Epitopes formed from contiguous residues (for example, amino acids, nucleotides, sugars, lipid moiety) typically are retained on exposure to denaturing solvents whereas epitopes formed by tertiary folding typically are lost on treatment with denaturing solvents. An epitope may include but is not limited to at least 3, at least 5 or 8-10 residues (for example, amino acids or nucleotides). In some examples an epitope is less than 20 residues (for example, amino acids or nucleotides) in length, less than 15 residues or less than 12 residues. Two antibodies may bind the same epitope within an antigen if they exhibit competitive binding for the antigen. In some embodiments, an epitope can be identified by a certain minimal distance to a CDR residue on the antigen-binding molecule. In some embodiments, an epitope can be identified by the above distance, and further limited to those residues involved in a bond (for example, a hydrogen bond) between an antibody residue and an antigen residue. An epitope can be identified by various scans as well, for example an alanine or arginine scan can indicate one or more residues that the antigen-binding molecule can interact with. Unless explicitly denoted, a set of residues as an epitope does not exclude other residues from being part of the epitope for a particular antibody. Rather, the presence of such a set designates a minimal series (or set of species) of epitopes. Thus, in some embodiments, a set of residues identified as an epitope designates a minimal epitope of relevance for the antigen, rather than an exclusive list of residues for an epitope on an antigen.

The term “antibody” herein is used in the broadest sense and encompasses various antibody structures, including but not limited to monoclonal antibodies, polyclonal antibodies, multispecific antibodies (for example, bispecific (such as Bi-specific T-cell engagers) and trispecific antibodies), and antibody fragments so long as they exhibit the desired antigen-binding activity.

The term antibody includes, but is not limited to, fragments that are capable of binding to an antigen, such as Fv, single-chain Fv (scFv), Fab, Fab′, di-scFv, sdAb (single domain antibody) and (Fab′)₂ (including a chemically linked F(ab′)₂). Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen. The term antibody also includes, but is not limited to, chimeric antibodies, humanized antibodies, and antibodies of various species such as mouse, human, cynomolgus monkey, etc. Furthermore, for all antibody constructs provided herein, variants having the sequences from other organisms are also contemplated. Thus, if a human version of an antibody is disclosed, one of skill in the art will appreciate how to transform the human sequence based antibody into a mouse, rat, cat, dog, horse, etc. sequence. Antibody fragments also include either orientation of single chain scFvs, tandem di-scFv, diabodies, tandem tri-sdcFv, minibodies, etc. Antibody fragments also include nanobodies (sdAb, an antibody having a single, monomeric domain, such as a pair of variable domains of heavy chains, without a light chain). An antibody fragment can be referred to as being a specific species in some embodiments (for example, human scFv or a mouse scFv). This denotes the sequences of at least part of the non-CDR regions, rather than the source of the construct.

The term “monoclonal antibody” refers to an antibody of a substantially homogeneous population of antibodies, that is, the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. Thus, a sample of monoclonal antibodies can bind to the same epitope on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

The term “CDR” denotes a complementarity determining region as defined by at least one manner of identification to one of skill in the art. In some embodiments, CDRs can be defined in accordance with any of the Chothia numbering schemes, the Kabat numbering scheme, a combination of Kabat and Chothia, the AbM definition, the contact definition, and/or a combination of the Kabat, Chothia, AbM, and/or contact definitions. Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991)). The AbM definition can include, for example, CDRs (CDR-L1, CDR-L2, CDR-L3, CDR-H1, CDR-H2, and CDR-H3) at amino acid residues 24-34 of L1, 50-56 of L2, 89-97 of L3, H26-H35B of H1, 50-58 of H2, and 95-102 of H3. The Contact definition can include, for example, CDRs (CDR-L1, CDR-L2, CDR-L3, CDR-H1, CDR-H2, and CDR-H3) at amino acid residues 30-36 of L1, 46-55 of L2, 89-96 of L3, 30-35 of H1, 47-58 of H2, and 93-101 of H3. The Chothia definition can include, for example, CDRs (CDR-L1, CDR-L2, CDR-L3, CDR-H1, CDR-H2, and CDR-H3) at amino acid residues 24-34 of L1, 50-56 of L2, 89-97 of L3, 26-32 . . . 34 of H1, 52-56 of H2, and 95-102 of H3. With the exception of CDR1 in V_{H}, CDRs generally comprise the amino acid residues that form the hypervariable loops. The various CDRs within an antibody can be designated by their appropriate number and chain type, including, without limitation as: a) CDR-L1, CDR-L2, CDR-L3, CDR-H1, CDR-H2, and CDR-H3; b) CDRL1, CDRL2, CDRL3, CDRH1, CDRH2, and CDRH3; c) LCDR-1, LCDR-2, LCDR-3, HCDR-1, HCDR-2, and HCDR-3; or d) LCDR1, LCDR2, LCDR3, HCDR1, HCDR2, and HCDR3; etc. The term “CDR” is used herein to also encompass HVR or a “hyper variable region”, including hypervariable loops. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).)

The term “heavy chain variable region” as used herein refers to a region comprising at least three heavy chain CDRs. In some embodiments, the heavy chain variable region includes the three CDRs and at least FR2 and FR3. In some embodiments, the heavy chain variable region includes at least heavy chain HCDR1, framework (FR) 2, HCDR2, FR3, and HCDR3. In some embodiments, a heavy chain variable region also comprises at least a portion of an FR1 and/or at least a portion of an FR4.

The term “heavy chain constant region” as used herein refers to a region comprising at least three heavy chain constant domains, C_{H}1, C_{H}2, and C_{H}3. Of course, non-function-altering deletions and alterations within the domains are encompassed within the scope of the term “heavy chain constant region,” unless designated otherwise. Nonlimiting exemplary heavy chain constant regions include γ, δ, and α. Nonlimiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a μ constant region is an IgM antibody, and an antibody comprising an ε constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

The term “heavy chain” as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence.

The term “light chain variable region” as used herein refers to a region comprising at least three light chain CDRs. In some embodiments, the light chain variable region includes the three CDRs and at least FR2 and FR3. In some embodiments, the light chain variable region includes at least light chain LCR1, framework (FR) 2, LCD2, FR3, and LCD3. For example, a light chain variable region may comprise light chain CDR1, framework (FR) 2, CDR2, FR3, and CDR3. In some embodiments, a light chain variable region also comprises at least a portion of an FR1 and/or at least a portion of an FR4.

The term “light chain constant region” as used herein refers to a region comprising a light chain constant domain, C_{L}. Nonlimiting exemplary light chain constant regions include λ and κ. Of course, non-function-altering deletions and alterations within the domains are encompassed within the scope of the term “light chain constant region,” unless designated otherwise.

The term “light chain” as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

“Affinity” refers to the strength of the sum total of noncovalent interactions between a single binding site of a molecule (for example, an antibody) and its binding partner (for example, an antigen). The affinity of a molecule X for its partner Y can generally be represented by the dissociation constant (K_{D}). Affinity can be measured by common methods known in the art (such as, for example, ELISA K_{D}, KinExA, bio-layer interferometry (BLI), and/or surface plasmon resonance devices (such as a BIAcore® device), including those described herein).

The term “K_{D}”, as used herein, refers to the equilibrium dissociation constant of an antibody-antigen interaction.

In some embodiments, the “K_{D},” “K_{d},” “Kd” or “Kd value” of the antibody is measured by using surface plasmon resonance assays using a BIACORE®-2000 or a BIACORE®-3000 (BIAcore, Inc., Piscataway, N.J.) at 25° C. with immobilized antigen CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μL/minute to achieve approximately 10 response units (RU) of coupled protein. Following the injection of antigen, 1 M ethanolamine is injected to block unreacted groups. For kinetics measurements, serial dilutions of polypeptide, for example, full length antibody, are injected in PBS with 0.05% TWEEN-20™ surfactant (PBST) at 25° C. at a flow rate of approximately 25 μL/min. Association rates (kₒₙ) and dissociation rates (Ur) are calculated using a simple one-to-one Langmuir binding model (BIACORE® Evaluation Software version 3.2) by simultaneously fitting the association and dissociation sensorgrams. The equilibrium dissociation constant (K_{d}) is calculated as the ratio k_{off}/kₒₙ. See, for example, Chen et al., J. Mol. Biol. 293:865-881 (1999). If the on-rate exceeds 10⁶ M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

“Surface plasmon resonance” denotes an optical phenomenon that allows for the analysis of real-time biospecific interactions by detection of alterations in protein concentrations within a biosensor matrix, for example using the BIAcore™ system (BIAcore International AB, a GE Healthcare company, Uppsala, Sweden and Piscataway, N.J.). For further descriptions, see Jonsson et al. (1993) Ann. Biol. Clin. 51:19-26.

“Biolayer interferometry” refers to an optical analytical technique that analyzes the interference pattern of light reflected from a layer of immobilized protein on a biosensor tip and an internal reference layer. Changes in the number of molecules bound to the biosensor tip cause shifts in the interference pattern that can be measured in real-time. A nonlimiting exemplary device for biolayer interferometry is ForteBio Octet® RED96 system (Pall Corporation). See, e.g., Abdiche et al., 2008, Anal. Biochem. 377: 209-277.

The term “biological activity” refers to any one or more biological properties of a molecule (whether present naturally as found in vivo, or provided or enabled by recombinant means). Biological properties include, but are not limited to, binding a receptor, inducing cell proliferation, inhibiting cell growth, inducing other cytokines, inducing apoptosis, and enzymatic activity. In some embodiments, biological activity of a PD-1 protein includes, for example, promoting apoptosis of antigen-specific T cells, reducing apoptosis of regulatory T (Treg) cells, inhibiting activation of T cells, inhibiting proliferation of T cells, and facilitating T cell anergy or exhaustion.

A “humanized antibody” as used herein refers to an antibody in which at least one amino acid in a framework region of a non-human variable region has been replaced with the corresponding amino acid from a human variable region. In some embodiments, a humanized antibody comprises at least one human constant region or fragment thereof. In some embodiments, a humanized antibody is an antibody fragment, such as Fab, an scFv, a (Fab′)₂, etc. The term humanized also denotes forms of non-human (for example, murine) antibodies that are chimeric immunoglobulins, immunoglobulin chains, or fragments thereof (such as Fv, Fab, Fab′, F(ab′)₂ or other antigen-binding subsequences of antibodies) that contain minimal sequence of non-human immunoglobulin. Humanized antibodies can include human immunoglobulins (recipient antibody) in which residues from a complementary determining region (CDR) of the recipient are substituted by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody can comprise residues that are found neither in the recipient antibody nor in the imported CDR or framework sequences, but are included to further refine and optimize antibody performance. In general, the humanized antibody can comprise substantially all of at least one, and typically two, variable domains, in which all or substantially all of the CDR regions correspond to those of a non-human immunoglobulin and all or substantially all of the FR regions are those of a human immunoglobulin consensus sequence. In some embodiments, the humanized antibody can also comprise at least a portion of an immunoglobulin constant region or domain (Fc), typically that of a human immunoglobulin. Other forms of humanized antibodies have one or more CDRs (CDR L1, CDR L2, CDR L3, CDR H1, CDR H2, and/or CDR H3) which are altered with respect to the original antibody, which are also termed one or more CDRs “derived from” one or more CDRs from the original antibody. As will be appreciated, a humanized sequence can be identified by its primary sequence and does not necessarily denote the process by which the antibody was created.

A “human antibody” as used herein encompasses antibodies produced in humans, antibodies produced in non-human animals that comprise human immunoglobulin genes, such as XenoMouse® mice, and antibodies selected using in vitro methods, such as phage display (Vaughan et al., 1996, Nature Biotechnology, 14:309-314; Sheets et al., 1998, Proc. Natl. Acad. Sci. (USA) 95:6157-6162; Hoogenboom and Winter, 1991, J. Mol. Biol., 227:381; Marks et al., 1991, J. Mol. Biol., 222:581), wherein the antibody repertoire is based on a human immunoglobulin sequence. The term “human antibody” denotes the genus of sequences that are human sequences. Thus, the term is not designating the process by which the antibody was created, but the genus of sequences that are relevant.

A “functional Fc region” possesses an “effector function” of a native sequence Fc region. Exemplary “effector functions” include Fc receptor binding; C1q binding; CDC; ADCC; phagocytosis; down regulation of cell surface receptors (for example B cell receptor; BCR), etc. Such effector functions generally require the Fc region to be combined with a binding domain (for example, an antibody variable domain) and can be assessed using various assays.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification. In some embodiments, a “variant Fc region” comprises an amino acid sequence which differs from that of a native sequence Fc region by virtue of at least one amino acid modification, yet retains at least one effector function of the native sequence Fc region. In some embodiments, the variant Fc region has at least one amino acid substitution compared to a native sequence Fc region or to the Fc region of a parent polypeptide, for example, from about one to about ten amino acid substitutions, and preferably, from about one to about five amino acid substitutions in a native sequence Fc region or in the Fc region of the parent polypeptide. In some embodiments, the variant Fc region herein will possess at least about 80% sequence identity with a native sequence Fc region and/or with an Fc region of a parent polypeptide, at least about 90% sequence identity therewith, at least about 95%, at least about 96%, at least about 97%, at least about 98%, or at least about 99% sequence identity therewith.

“Fc receptor” or “FcR” describes a receptor that binds to the Fc region of an antibody. In some embodiments, an FcγR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (see, for example, Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, for example, Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

“Effector functions” refer to biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (for example B cell receptor); and B cell activation.

“Human effector cells” are leukocytes which express one or more FcRs and perform effector functions. In some embodiments, the cells express at least FcγRIII and perform ADCC effector function(s). Examples of human leukocytes which mediate ADCC include peripheral blood mononuclear cells (PBMC), natural killer (NK) cells, monocytes, cytotoxic T cells, and neutrophils. The effector cells may be isolated from a native source, for example, from blood.

“Antibody-dependent cell-mediated cytotoxicity” or “ADCC” refers to a form of cytotoxicity in which secreted Ig bound onto Fc receptors (FcRs) present on certain cytotoxic cells (for example NK cells, neutrophils, and macrophages) enable these cytotoxic effector cells to bind specifically to an antigen-bearing target cell and subsequently kill the target cell with cytotoxins. The primary cells for mediating ADCC, NK cells, express FcγRIII only, whereas monocytes express FcγRT, FcγRII, and FcγRIII FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. Nos. 5,500,362 or 5,821,337 or 6,737,056 (Presta), may be performed. Useful effector cells for such assays include PBMC and NK cells. Alternatively, or additionally, ADCC activity of the molecule of interest may be assessed in vivo, for example, in an animal model such as that disclosed in Clynes et al. Proc. Natl. Acad. Sci. (USA) 95:652-656 (1998). Additional polypeptide variants with altered Fc region amino acid sequences (polypeptides with a variant Fc region) and increased or decreased ADCC activity are described, for example, in U.S. Pat. Nos. 7,923,538, and 7,994,290.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, for example, as described in Gazzano-Santoro et al., J. Immunol. Methods 202:163 (1996), may be performed. Polypeptide variants with altered Fc region amino acid sequences (polypeptides with a variant Fc region) and increased or decreased C1q binding capability are described, for example, in U.S. Pat. No. 6,194,551 B1, U.S. Pat. Nos. 7,923,538, 7,994,290 and WO 1999/51642. See also, for example, Idusogie et al., J. Immunol. 164: 4178-4184 (2000).

A polypeptide variant with “altered” FcR binding affinity or ADCC activity is one which has either enhanced or diminished FcR binding activity and/or ADCC activity compared to a parent polypeptide or to a polypeptide comprising a native sequence Fc region. The polypeptide variant which “displays increased binding” to an FcR binds at least one FcR with better affinity than the parent polypeptide. The polypeptide variant which “displays decreased binding” to an FcR, binds at least one FcR with lower affinity than a parent polypeptide. Such variants which display decreased binding to an FcR may possess little or no appreciable binding to an FcR, for example, 0-20% binding to the FcR compared to a native sequence IgG Fc region.

The polypeptide variant which “mediates antibody-dependent cell-mediated cytotoxicity (ADCC) in the presence of human effector cells more effectively” than a parent antibody is one which in vitro or in vivo is more effective at mediating ADCC, when the amounts of polypeptide variant and parent antibody used in the assay are essentially the same. Generally, such variants will be identified using the in vitro ADCC assay as herein disclosed, but other assays or methods for determining ADCC activity, for example in an animal model etc., are contemplated.

The term “substantially similar” or “substantially the same,” as used herein, denotes a sufficiently high degree of similarity between two or more numeric values such that one of skill in the art would consider the difference between the two or more values to be of little or no biological and/or statistical significance within the context of the biological characteristic measured by said value. In some embodiments the two or more substantially similar values differ by no more than about any one of 5%, 10%, 15%, 20%, 25%, or 50%.

The phrase “substantially different,” as used herein, denotes a sufficiently high degree of difference between two numeric values such that one of skill in the art would consider the difference between the two values to be of statistical significance within the context of the biological characteristic measured by said values. In some embodiments, the two substantially different numeric values differ by greater than about any one of 10%, 20%, 25%, 30%, 35%, 40%, 45%, 50%, 60%, 70%, 80%, 90%, or 100%.

The phrase “substantially reduced,” as used herein, denotes a sufficiently high degree of reduction between a numeric value and a reference numeric value such that one of skill in the art would consider the difference between the two values to be of statistical significance within the context of the biological characteristic measured by said values. In some embodiments, the substantially reduced numeric values is reduced by greater than about any one of 10%, 20%, 25%, 30%, 35%, 40%, 45%, 50%, 60%, 70%, 80%, 90%, or 100% compared to the reference value.

As used herein, “Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 1. Amino acid substitutions may be introduced into an antibody of interest and the products screened for a desired activity, for example, retained/improved antigen binding, decreased immunogenicity, or improved ADCC or CDC.

TABLE 1

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The terms “individual” or “subject” are used interchangeably herein to refer to an animal; for example a mammal. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder.

The term “sample” or “patient sample” as used herein, refers to a composition that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. By “tissue or cell sample” is meant a collection of similar cells obtained from a tissue of a subject or patient. The source of the tissue or cell sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

A “reference” as used herein, refers to any sample, standard, or level that is used for comparison purposes.

A “reference sample”, “reference cell”, or “reference tissue”, as used herein, refers to a sample, cell or tissue obtained from a source known, or believed, not to be afflicted with the disease or condition for which a method or composition of the invention is being used to identify. In some embodiments, a reference sample, reference cell or reference tissue is obtained from a healthy part of the body of the same subject or patient in whom a disease or condition is being identified using a composition or method of the invention. In some embodiments, a reference sample, reference cell or reference tissue is obtained from a healthy part of the body of one or more individuals who are not the subject or patient in whom a disease or condition is being identified using a composition or method of the invention.

A “disease” or “disorder” as used herein refers to a condition where treatment is needed and/or desired.

“Cancer” and “tumor,” as used herein, are interchangeable terms that refer to any abnormal cell or tissue growth or proliferation in an animal. As used herein, the terms “cancer” and “tumor” encompass solid and hematological/lymphatic cancers and also encompass malignant, pre-malignant, and benign growth, such as dysplasia. Examples of cancer include but are not limited to, carcinoma, lymphoma, blastoma, sarcoma, and leukemia. More particular non-limiting examples of such cancers include lung cancer, small-cell lung cancer (SCLC), non-small cell lung cancer (NSCLC), uretheral cancer, squamous cell cancer, small-cell lung cancer, pituitary cancer, esophageal cancer, astrocytoma, soft tissue sarcoma, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma (including uterine corpus endometrial carcinoma), salivary gland carcinoma, kidney cancer, renal cancer, liver cancer, prostate cancer, vulval cancer, thyroid cancer, hepatic carcinoma, brain cancer, testis cancer, cholangiocarcinoma, gallbladder carcinoma, gastric cancer, melanoma, mesothelioma, and various types of head and neck cancer.

As used herein, “treatment” is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

“Ameliorating” means a lessening or improvement of one or more symptoms as compared to not administering an anti-PD-1 antibody. “Ameliorating” also includes shortening or reduction in duration of a symptom.

In the context of cancer, the term “treating” includes any or all of: inhibiting growth of cancer cells, inhibiting replication of cancer cells, lessening of overall tumor burden and ameliorating one or more symptoms associated with the disease.

The term “biological sample” means a quantity of a substance from a living thing or formerly living thing. Such substances include, but are not limited to, blood, (for example, whole blood), plasma, serum, urine, amniotic fluid, synovial fluid, endothelial cells, leukocytes, monocytes, other cells, organs, tissues, bone marrow, lymph nodes and spleen.

The term “control” refers to a composition known to not contain an analyte (“negative control”) or to contain analyte (“positive control”). A positive control can comprise a known concentration of analyte. “Control,” “positive control,” and “calibrator” may be used interchangeably herein to refer to a composition comprising a known concentration of analyte. A “positive control” can be used to establish assay performance characteristics and is a useful indicator of the integrity of reagents (for example, analytes).

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some embodiments, by “reduce” or “inhibit” is meant the ability to cause an overall decrease of 20% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control dose (such as a placebo) over the same period of time. Unless otherwise specified, the terms “reduce”, “inhibit”, or “prevent” do not denote or require complete prevention over all time.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. Unless otherwise specified, the terms “reduce”, “inhibit”, or “prevent” do not denote or require complete prevention over all time.

As used herein, to “suppress” a function or activity is to reduce the function or activity when compared to otherwise same conditions except for a condition or parameter of interest, or alternatively, as compared to another condition. For example, an antibody which suppresses tumor growth reduces the rate of growth of the tumor compared to the rate of growth of the tumor in the absence of the antibody.

A “therapeutically effective amount” of a substance/molecule, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at doses and for periods of time necessary, to achieve the desired therapeutic and/or prophylactic result.

A “prophylactically effective amount” refers to an amount effective, at doses and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The terms “pharmaceutical formulation” and “pharmaceutical composition” refer to a preparation which is in such form as to permit the biological activity of the active ingredient(s) to be effective, and which contains no additional components which are unacceptably toxic to a subject to which the formulation would be administered. Such formulations may be sterile. A “sterile” formulation is aseptic or essentially free from living microorganisms and their spores.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the doses and concentrations employed and is compatible with other ingredients of the formulation. The pharmaceutically acceptable carrier is appropriate for the formulation employed.

Administration “in combination with” one or more further therapeutic agents includes simultaneous (concurrent) and consecutive or sequential administration in any order.

The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about a specified number of minutes.

The term “sequentially” is used herein to refer to administration of two or more therapeutic agents where the administration of one or more agent(s) continues after discontinuing the administration of one or more other agent(s), or wherein administration of one or more agent(s) begins before the administration of one or more other agent(s). For example, administration of the two or more therapeutic agents are administered with a time separation of more than about a specified number of minutes.

As used herein, “in conjunction with” refers to administration of one treatment modality in addition to another treatment modality. As such, “in conjunction with” refers to administration of one treatment modality before, during or after administration of the other treatment modality to the individual.

The term “package insert” is used to refer to instructions customarily included in commercial packages of therapeutic products, that contain information about the indications, usage, dose, administration, combination therapy, contraindications and/or warnings concerning the use of such therapeutic products. These are also referred to as the Full Prescribing Information for a product in the U.S.

An “article of manufacture” is any manufacture (for example, a package or container) or kit comprising at least one reagent, for example, a medicament for treatment of a disease or disorder (for example, cancer), or a probe for specifically detecting a biomarker described herein. In some embodiments, the manufacture or kit is promoted, distributed, or sold as a unit for performing the methods described herein.

### II. Therapeutic Methods

Methods of treating disease in a subject in need of such treatment comprising administering an anti-PD-1 antibody. Nonlimiting exemplary diseases that can be treated with anti-PD-1 antibodies include, but are not limited to, cancer. Determination of the frequency of administration can be made by persons skilled in the art, such as an attending physician based on considerations of the condition being treated, age of the subject being treated, severity of the condition being treated, general state of health of the subject being treated and the like. In some embodiments, anti-PD-1 antibodies are administered in an amount effective for treatment of (including prophylaxis of) cancer. The therapeutically effective amount is typically dependent on the weight of the subject being treated, his or her physical or health condition, the extensiveness of the condition to be treated, or the age of the subject being treated, pharmaceutical formulation methods, and/or administration methods (e.g., administration time and administration route).

In some embodiments, a method of treating cancer in a subject comprises administering an anti-PD-1 antibody to the subject periodically. In some embodiments, a dose of the anti-PD-1 antibody is administered every 2 weeks, every 3 weeks, every 4 weeks, every 5 weeks, every 6 weeks, every 7 weeks, every 8 weeks, every 9 weeks, every 10 weeks, every 11 weeks, or every 12 weeks.

In some embodiments, the method comprises administering 1, 2, 3, 6, 9, 18 doses of the anti-PD-1 antibody in a period of about 18 weeks. In some embodiments, the method comprises administering 6 doses of the anti-PD-1 antibody in a period of 18 weeks. In some embodiments, the method comprises administering 3 doses of the anti-PD-1 antibody in a period of 18 weeks.

In some embodiments, a method of treating cancer in a subject comprises administering an anti-PD-1 antibody to the subject, wherein a dose of the anti-PD-1 antibody is administered once every 6 weeks. In some embodiments, a method of treating cancer in a subject comprises administering an anti-PD-1 antibody to the subject, wherein a dose of the anti-PD-1 antibody is administered once every 5 weeks. In some embodiments, a method of treating cancer in a subject comprises administering an anti-PD-1 antibody to the subject, wherein a dose of the anti-PD-1 antibody is administered once every 4 weeks. In some embodiments, a method of treating cancer in a subject comprises administering an anti-PD-1 antibody to the subject, wherein a dose of the anti-PD-1 antibody is administered once every 3 weeks. In some embodiments, a method of treating cancer in a subject comprises administering an anti-PD-1 antibody to the subject, wherein a dose of the anti-PD-1 antibody is administered once every 2 weeks. In some embodiments, a method of treating cancer in a subject comprises administering an anti-PD-1 antibody to the subject, wherein a dose of the anti-PD-1 antibody is administered once every 1 week.

In some embodiments, each dose of the anti-PD-1 antibody is from about 100 mg/kg to about 1500 mg/kg. In some embodiments, each dose of the anti-PD-1 antibody is 100, 200, 300, 400, 500, 600, 700, 800, 900, 1000, 1100, 1200, 1300, 1400, or 1500 mg/kg.

In some embodiments, a method of treating cancer in a subject comprises administering an anti-PD-1 antibody to the subject, wherein a dose of the anti-PD-1 antibody at 1000 mg/kg is administered once every 6 weeks. In some embodiments, a method of treating cancer in a subject comprises administering an anti-PD-1 antibody to the subject, wherein a dose of the anti-PD-1 antibody at 400-600 mg/kg is administered once every 3 weeks.

In some embodiments, a method of treating cancer in a subject comprises administering a dose of an anti-PD-1 antibody to the subject at 1000 mg/kg once every 6 weeks. In some embodiments, a method of treating cancer in a subject comprises administering a dose of an anti-PD-1 antibody to the subject at 400-600 mg/kg once every 3 weeks. In some embodiments, a dose of the anti-PD-1 antibody at 400 mg/kg is administered once every 3 weeks. In some embodiments, a dose of the anti-PD-1 antibody at 500 mg/kg is administered once every 3 weeks. In some embodiments, a dose of the anti-PD-1 antibody at 600 mg/kg is administered once every 3 weeks.

In some embodiments, the subject is administered the anti-PD-1 antibody for a period of about 12 weeks. In some embodiments, the subject is administered the anti-PD-1 antibody for a period of about 18 weeks. In some embodiments, the subject is administered the anti-PD-1 antibody for a period of about 24 weeks. In some embodiments, the subject is administered the anti-PD-1 antibody for a period of about 36 weeks. In some embodiments, the subject is administered the anti-PD-1 antibody for a period of about 48 weeks. In some embodiments, the subject is administered the anti-PD-1 antibody for a period of about 52 weeks.

In some embodiments, a method of enhancing an immune response in a subject is provided, comprising administering a dose of an anti-PD-1 antibody to the subject at 1000 mg/kg once every 6 weeks. In some embodiments, a method of enhancing an immune response in a subject is provided, comprising administering a dose of an anti-PD-1 antibody to the subject at 400-600 mg/kg once every 3 weeks.

In some embodiments, a method of increasing activation of a T cell in a mammal is provided, comprising administering a dose of an anti-PD-1 antibody to the subject at 1000 mg/kg once every 6 weeks. In some embodiments, a method of increasing activation of a T cell in a mammal is provided, comprising administering a dose of an anti-PD-1 antibody to the subject at 400-600 mg/kg once every 3 weeks.

In some embodiments, a method of reducing tumor size in a in a mammal with cancer is provided, comprising administering a dose of an anti-PD-1 antibody to the subject at 1000 mg/kg once every 6 weeks. In some embodiments, a method of reducing tumor size in a in a mammal with cancer is provided, comprising administering a dose of an anti-PD-1 antibody to the subject at 400-600 mg/kg once every 3 weeks.

a. Subject

Subjects or Patients that can be treated as described herein are patients having a cancer. The type of cancer can be any type of cancer listed herein or otherwise known in the art. Exemplary types of cancer include, but are not limited to, melanoma, non-small cell lung cancer (NSCLC), small cell lung cancer (SCLC), renal cell carcinoma (RCC) (e.g., clear cell RCC), gastric cancer, bladder cancer, endometrial cancer, MSI-H cancer of any organ, diffuse large B-cell lymphoma (DLBCL), Hodgkin's lymphoma, ovarian cancer (e.g., endometrioid ovarian cancer), head and neck squamous cell cancer (HNSCC), acute myeloid leukemia (AML), rectal cancer, refractory testicular cancer, small cell lung cancer (SCLC), small bowel cancer, metastatic cutaneous squamous cell cancer, cervical cancer, MSI-high colon cancer, esophageal cancer, mesothelioma, breast cancer, and triple negative breast cancer (TNBC). Also see the definition of cancer, above, for additional cancer types that can be treated according to the methods of the invention.

In some embodiments, a method of treating cancer is provided, wherein cells within a sample of the tumor express PD-L1. In some such embodiments, the tumor may be considered to be PD-L1-positive, or to express PD-L1. Expression of PD-L1 may be determined by IHC, e.g., as discussed herein. In some embodiments, a tumor is considered to express PD-L1 when a sample from the tumor shows 1+, 2+, or 3+ staining of PD-L1 by IHC. In some embodiments, the sample from the tumor shows 2+ or 3+ staining of PD-L1 by IHC. In some embodiments, a tumor sample from a subject is analyzed for PD-L1 expression and the subject is selected for treatment with an antibody described herein if the tumor sample shows PD-L1 expression. In some embodiments, the subject is selected if the tumor sample shows elevated expression of PD-L1.

In some embodiments, a subject is selected for treatment with an anti-PD-1 antibody provided herein if the subject's tumor is PD-L1^{HIGH}. In some embodiments, a subject is selected for treatment with an anti-PD-1 antibody provided herein if the subject's tumor is PD-L1^{LOW}. In some embodiments, a subject is selected for treatment with an anti-PD-1 antibody provided herein if the subject's tumor is PD-1^{HIGH}/PD-L1^{LOW}. In some embodiments, a subject is selected for treatment with an anti-PD-1 antibody provided herein if the subject's tumor is PD-1^{HIGH}/PD-L1^{HIGH}.

Patients that can be treated as described herein include patients who have not previously received an anti-cancer therapy and patients who have received previous (e.g., 1, 2, 3, 4, 5, or more) doses or cycles of one or more (e.g., 1, 2, 3, 4, 5, or more) anti-cancer therapies.

b. Pharmaceutical Compositions

In some embodiments, compositions comprising anti-PD-1 antibodies are provided in formulations with a wide variety of pharmaceutically acceptable carriers (see, for example, Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7^{th} ed., Lippencott Williams and Wilkins (2004); Kibbe et al., Handbook of Pharmaceutical Excipients, 3^{rd} ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Non-limiting exemplary carriers include saline, buffered saline, dextrose, water, glycerol, ethanol, and combinations thereof.

In some embodiments, a pharmaceutical composition comprising an anti-PD-1 antibody is provided for the methods described herein. In some embodiments, the pharmaceutical composition comprises a chimeric antibody. In some embodiments, the pharmaceutical composition comprises a humanized antibody. In some embodiments, the pharmaceutical composition comprises an antibody prepared in a host cell or cell-free system as described herein. In some embodiments, the pharmaceutical composition comprises pharmaceutically acceptable carrier.

In some embodiments, pharmaceutical compositions are administered in an amount effective for treatment of (including prophylaxis of) cancer. The therapeutically effective amount is typically dependent on the weight of the subject being treated, his or her physical or health condition, the extensiveness of the condition to be treated, or the age of the subject being treated.

### Routes of Administration

In some embodiments, anti-PD-1 antibodies and/or additional therapeutic agents can be administered in vivo by various routes, including, but not limited to, intravenous, intra-arterial, parenteral, intratumoral, intraperitoneal or subcutaneous. The appropriate formulation and route of administration may be selected according to the intended application.

### III. Anti-PD-1 Antibodies

Antibodies directed against PD-1 are provided. Anti-PD-1 antibodies include, but are not limited to, humanized antibodies, chimeric antibodies, mouse antibodies, human antibodies, and antibodies comprising the heavy chain and/or light chain CDRs discussed herein. In some embodiments, an isolated antibody that binds to PD-1 is provided. In some embodiments, a monoclonal antibody that binds to PD-1 is provided. In some embodiments, an anti-PD-1 antibody is an anti-PD-1 antagonist antibody. In some embodiments, an anti-PD-1 antibody provided herein inhibits binding of PD-1 to PD-L1 and/or PD-L2. In some embodiments, an anti-PD-1 antibody provided herein inhibits binding of PD-1 to PD-L1. In some embodiments, an anti-PD-1 antibody provided herein inhibits binding of PD-1 to PD-L1 and PD-L2. In some embodiments, administration of the anti-PD-1 antibodies described herein enhances an immune response in a subject, and/or increases activation of T cells in a subject.

Exemplary anti-PD-1 antibodies are further described, e.g., in WO 2018/085358, the content of which is herein incorporated in its entirety by reference. In some embodiments, the anti-PD-1 antibody is JTX-4014 (Jounce Therapeutics; WO 2018/085358). Other PD-1 antibodies include nivolumab (anti-PD-1 antibody; BMS-936558, MDX-1106, ONO-4538; OPDIVO®; Bristol-Myers Squibb); pidilizumab (anti-PD-1 antibody, CureTech), pembrolizumab (anti-PD-1 antibody; KEYTRUDA®, MK-3475, lambrolizumab); durvalumab (anti-PD-L1 antibody, MEDI-4736; AstraZeneca/MedImmune); RG-7446; MSB-0010718C; AMP-224; BMS-936559 (an anti-PD-L1 antibody; Bristol-Myers Squibb); AMP-514; MDX-1105; ANB-011; anti-LAG-3/PD-1; anti-PD-1 Ab (CoStim); anti-PD-1 Ab (Kadmon Pharm.); anti-PD-1 Ab (Immunovo); and anti-TIM-3/PD-1 Ab (AnaptysBio).

In some embodiments, the anti-PD-1 antibody binds to PD-1 and inhibits binding of PD-1 to PD-L1 and/or PD-L2. In some embodiments, the anti-PD-1 antibody binds to PD-1 and enhances an immune response in a subject, and/or increases activation of T cells in a subject following administration of the antibody to the subject.

In certain preferred embodiments, the anti-PD-1 antibody is an antibody having light and heavy chain sequences corresponding to SEQ ID NOs: 28 and 29, respectively. In some embodiments, the anti-PD-1 antibody is JTX-4014.

In some embodiments, an anti-PD-1 antibody comprises (a) HCDR1 comprising the amino acid sequence of SEQ ID NO: 21; (b) HCDR2 comprising the amino acid sequence of SEQ ID NO: 22; (c) HCDR3 comprising the amino acid sequence of SEQ ID NO: 23; (d) LCDR1 comprising the amino acid sequence of SEQ ID NO: 25; (e) LCDR2 comprising the amino acid sequence of SEQ ID NO: 26; and (f) LCDR3 comprising the amino acid sequence of SEQ ID NO: 27.

In some embodiments, an anti-PD-1 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 20, and a light chain variable domain (VL) having at least 90%, 91%, 92%, 93%, 94%, 95%, 96%, 97%, 98%, 99%, or 100% sequence identity to the amino acid sequence of SEQ ID NO: 24. In some embodiments, a VH sequence having at least 90%, 91%, 92%, 93%, 94%, 95%, 96%, 97%, 98%, or 99% identity contains substitutions (for example, conservative substitutions), insertions, or deletions relative to the reference sequence, and a VL sequence having at least 90%, 91%, 92%, 93%, 94%, 95%, 96%, 97%, 98%, or 99% identity contains substitutions (for example, conservative substitutions), insertions, or deletions relative to the reference sequence, but an anti-PD-1 antibody comprising that sequence retains the ability to bind to PD-1. In some embodiments, a total of 1 to 10 amino acids have been substituted, inserted and/or deleted in SEQ ID NO: 20. In some embodiments, a total of 1 to 10 amino acids have been substituted, inserted and/or deleted in SEQ ID NO: 24. In some embodiments, substitutions, insertions, or deletions occur in regions outside the CDRs (that is, in the FRs). In some embodiments, the anti-PD-1 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 20, and a light chain variable domain (VL) having at least 90%, 91%, 92%, 93%, 94%, 95%, 96%, 97%, 98%, 99%, or 100% sequence identity to the amino acid sequence of SEQ ID NO: 24; wherein the antibody comprises (a) HCDR1 comprising the amino acid sequence of SEQ ID NO: 21; (b) HCDR2 comprising the amino acid sequence of SEQ ID NO: 22; (c) HCDR3 comprising the amino acid sequence of SEQ ID NO: 23; (d) LCDR1 comprising the amino acid sequence of SEQ ID NO: 25; (e) LCDR2 comprising the amino acid sequence of SEQ ID NO: 26; and (f) LCDR3 comprising the amino acid sequence of SEQ ID NO: 27.

In some embodiments, the anti-PD-1 antibody comprises the VH sequence in SEQ ID NO: 20 and the VL sequence of SEQ ID NO: 24, including post-translational modifications of one or both sequences. In some embodiments, the anti-PD-1 antibody comprises the heavy chain sequence in SEQ ID NO: 28 and the light chain sequence of SEQ ID NO: 29, including post-translational modifications of one or both sequences.

In some embodiments, an anti-PD-1 antibody is provided that competes with an anti-PD-1 antibody described herein for binding to PD-1.

### IV. Antibody Expression and Production

### Nucleic Acid Molecules Encoding Anti-PD-1 Antibodies

Nucleic acid molecules comprising polynucleotides that encode one or more chains of an anti-PD-1 antibody are provided herein. In some embodiments, a nucleic acid molecule comprises a polynucleotide that encodes a heavy chain or a light chain of an anti-PD-1 antibody. In some embodiments, a nucleic acid molecule comprises both a polynucleotide that encodes a heavy chain and a polynucleotide that encodes a light chain, of an anti-PD-1 antibody. In some embodiments, a first nucleic acid molecule comprises a first polynucleotide that encodes a heavy chain and a second nucleic acid molecule comprises a second polynucleotide that encodes a light chain.

In some embodiments, the heavy chain and the light chain are expressed from one nucleic acid molecule, or from two separate nucleic acid molecules, as two separate polypeptides. In some embodiments, such as when an antibody is an scFv, a single polynucleotide encodes a single polypeptide comprising both a heavy chain and a light chain linked together.

In some embodiments, a polynucleotide encoding a heavy chain or light chain of an anti-PD-1 antibody comprises a nucleotide sequence that encodes at least one of the CDRs provided herein. In some embodiments, a polynucleotide encoding a heavy chain or light chain of an anti-PD-1 antibody comprises a nucleotide sequence that encodes at least 3 of the CDRs provided herein. In some embodiments, a polynucleotide encoding a heavy chain or light chain of an anti-PD-1 antibody comprises a nucleotide sequence that encodes at least 6 of the CDRs provided herein. In some embodiments, a polynucleotide encoding a heavy chain or light chain of an anti-PD-1 antibody comprises a nucleotide sequence that encodes a leader sequence, which, when translated, is located at the N terminus of the heavy chain or light chain. As discussed above, the leader sequence may be the native heavy or light chain leader sequence, or may be another heterologous leader sequence.

In some embodiments, the nucleic acid is one that encodes for any of the amino acid sequences for the antibodies in the Sequence Table herein. In some embodiments, the nucleic acid is one that is at least 80% identical to a nucleic acid encoding any of the amino acid sequences for the antibodies in the Sequence Table herein, for example, at least 80, 85, 90, 91, 92, 93, 94, 95, 96, 97, 98, or 99% identical. In some embodiments, the nucleic acid is one that hybridizes to any one or more of the nucleic acid sequences provided herein. In some of the embodiments, the hybridization is under moderate conditions. In some embodiments, the hybridization is under highly stringent conditions, such as: at least about 6×SSC and 1% SDS at 65° C., with a first wash for 10 minutes at about 42° C. with about 20% (v/v) formamide in 0.1×SSC, and with a subsequent wash with 0.2×SSC and 0.1% SDS at 65° C.

Nucleic acid molecules can be constructed using recombinant DNA techniques conventional in the art. In some embodiments, a nucleic acid molecule is an expression vector that is suitable for expression in a selected host cell.

Vectors comprising polynucleotides that encode anti-PD-1 heavy chains and/or anti-PD-1 light chains are provided. Vectors comprising polynucleotides that encode anti-PD-1 heavy chains and/or anti-PD-1 light chains are also provided. Such vectors include, but are not limited to, DNA vectors, phage vectors, viral vectors, retroviral vectors, etc. In some embodiments, a vector comprises a first polynucleotide sequence encoding a heavy chain and a second polynucleotide sequence encoding a light chain. In some embodiments, the heavy chain and light chain are expressed from the vector as two separate polypeptides. In some embodiments, the heavy chain and light chain are expressed as part of a single polypeptide, such as, for example, when the antibody is an scFv.

In some embodiments, a first vector comprises a polynucleotide that encodes a heavy chain and a second vector comprises a polynucleotide that encodes a light chain. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

In some embodiments, a vector is selected that is optimized for expression of polypeptides in CHO or CHO-derived cells, or in NSO cells. Exemplary such vectors are described, for example, in Running Deer et al., Biotechnol. Prog. 20:880-889 (2004).

### Host Cells

In some embodiments, anti-PD-1 antibody heavy chains and/or anti-PD-1 antibody light chains may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6® cells (Crucell); and NSO cells. In some embodiments, anti-PD-1 antibody heavy chains and/or anti-PD-1 antibody light chains may be expressed in yeast. See, for example, U.S. Publication No. US 2006/0270045 A1. In some embodiments, a particular eukaryotic host cell is selected based on its ability to make desired post-translational modifications to the anti-PD-1 antibody heavy chains and/or anti-PD-1 antibody light chains. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc. Nonlimiting exemplary methods are described, for example, in Sambrook et al., Molecular Cloning, A Laboratory Manual, 3rd ed. Cold Spring Harbor Laboratory Press (2001). Nucleic acids may be transiently or stably transfected in the desired host cells, according to any suitable method.

Host cells comprising any of the polynucleotides or vectors described herein are also provided. In some embodiments, a host cell comprising an anti-PD-1 antibody is provided. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtilis) and yeast (such as S. cerevisae, S. pombe; or K. lactis).

### Purification of Antibodies

Anti-PD-1 antibodies can be purified by any suitable method. Such methods include, but are not limited to, the use of affinity matrices or hydrophobic interaction chromatography. Suitable affinity ligands include the ROR1 ECD and ligands that bind antibody constant regions. For example, a Protein A, Protein G, Protein A/G, or an antibody affinity column may be used to bind the constant region and to purify an anti-PD-1 antibody. Hydrophobic interactive chromatography, for example, a butyl or phenyl column, may also suitable for purifying some polypeptides such as antibodies. Ion exchange chromatography (for example anion exchange chromatography and/or cation exchange chromatography) may also suitable for purifying some polypeptides such as antibodies. Mixed-mode chromatography (for example reversed phase/anion exchange, reversed phase/cation exchange, hydrophilic interaction/anion exchange, hydrophilic interaction/cation exchange, etc.) may also suitable for purifying some polypeptides such as antibodies. Many methods of purifying polypeptides are known in the art.

### Cell-Free Production of Antibodies

In some embodiments, an anti-PD-1 antibody is produced in a cell-free system. Nonlimiting exemplary cell-free systems are described, for example, in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

### Compositions

In some embodiments, antibodies prepared by the methods described above are provided. In some embodiments, the antibody is prepared in a host cell. In some embodiments, the antibody is prepared in a cell-free system. In some embodiments, the antibody is purified. In some embodiments, the antibody prepared in a host cell or a cell-free system is a chimeric antibody. In some embodiments, the antibody prepared in a host cell or a cell-free system is a humanized antibody. In some embodiments, the antibody prepared in a host cell or a cell-free system is a human antibody. In some embodiments, a cell culture media comprising an anti-PD-1 antibody is provided. In some embodiments, a host cell culture fluid comprising an anti-PD-1 antibody is provided.

In some embodiments, compositions comprising antibodies prepared by the methods described above are provided. In some embodiments, the composition comprises an antibody prepared in a host cell. In some embodiments, the composition comprises an antibody prepared in a cell-free system. In some embodiments, the composition comprises a purified antibody. In some embodiments, the composition comprises a chimeric antibody prepared in a host cell or a cell-free system. In some embodiments, the composition comprises a humanized antibody prepared in a host cell or a cell-free system. In some embodiments, the composition comprises a human antibody prepared in a host cell or a cell-free system.

In some embodiments, a composition comprising anti-PD-1 antibody at a concentration of more than about any one of 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 125 mg/mL, 150 mg/mL, 175 mg/mL, 200 mg/mL, 225 mg/mL, or 250 mg/mL is provided. In some embodiments, the composition comprises a chimeric antibody prepared in a host cell or a cell-free system. In some embodiments, the composition comprises a humanized antibody prepared in a host cell or a cell-free system. In some embodiments, the composition comprises a human antibody prepared in a host cell or a cell-free system.

### V. Combination Therapy

Anti-PD-1 antibodies can be administered alone or with other modes of treatment. They can be provided before, substantially contemporaneous with, and/or after other modes of treatment, for example, surgery, chemotherapy, radiation therapy, or the administration of a biologic, such as another therapeutic antibody.

In some embodiments, a method of treating cancer in a subject is provided, comprising administering an anti-PD-1 antibody and at least one additional therapeutic agent. In some such embodiments, the additional therapeutic agent is administered concurrently and/or sequentially with the anti-PD-1 antibody. In some embodiments, the additional therapeutic agent is selected from an anti-ICOS antibody, an anti-CTLA4 antibody, an OX40 antibody, a TIGIT antibody, an IDO inhibitor, an RORγ agonist, a chemotherapy, and a cancer vaccine, each of which is further described herein. In some embodiments, the additional therapeutic agent is an anti-ICOS antibody.

In some embodiments, a method of treating cancer in a subject is provided, comprising administering an anti-PD-1 antibody and an anti-ICOS antibody. In some embodiments, a method of treating cancer in a subject is provided, comprising administering an anti-PD-1 antibody and an anti-CTLA4 antibody. In some embodiments, a method of treating cancer in a subject is provided, comprising administering an anti-PD-1 antibody and an anti-OX40 antibody.

In some embodiments, the additional therapeutic agent is administered concurrently or sequentially with the anti-PD-1 antibody. In some embodiments, the first dose of the anti-PD-1 antibody is administered after the first dose of the at least one additional therapeutic agent. In some embodiments, the first dose of the anti-PD-1 antibody is administered before the first dose of the at least one additional therapeutic agent. In some embodiments, the first dose of the anti-PD-1 antibody is administered 1, 2, 3, 4, 5, or 6 weeks after the first dose of the additional therapeutic agent. In some embodiments, a method of treating cancer in a subject comprises administering multiple doses of an anti-PD-1 antibody to the subject and administering multiple doses of the at least one additional therapeutic agent. In some embodiments, the method comprises administering more doses of the anti-PD-1 antibody than the at least one additional therapeutic agent. In some embodiments, the method comprises administering fewer doses of the anti-PD-1 antibody than the at least one additional therapeutic agent.

As examples, one or more additional anti-cancer therapies discussed herein or otherwise known in the art, can be used in connection with the methods described herein. Exemplary additional anti-cancer therapies are described below.

a. Anti-ICOS Antibodies

In some embodiments, an anti-PD-1 antibody is administered in combination with an anti-ICOS antibody, such as an anti-ICOS agonist antibody. An anti-ICOS antibody refers to an agent capable of inhibiting the activity of inducible T-cell costimulatory (ICOS), thereby activating the immune system. The anti-ICOS antibody may bind to ICOS and increases the number of Teff cells and/or activates Teff cells and/or decreases Treg cells in a subject; and/or increases the ratio of Teff cells to Treg cells.

In some embodiments, the anti-ICOS antibody is an agonist antibody. See WO 2016/154177 and WO 2017/070423, which are each specifically incorporated herein by reference. Exemplary anti-ICOS antibodies include, but are not limited to, vopratelimab (Jounce Therapeutics; US 2016/0304610; WO 2016/154177; WO 2017/070423); GSK-3359069 (GSK); KY1044 (Kymab); KY1055 (Kymab); and BMS-986226 (Bristol-Myers Squibb). In some embodiments, the anti-ICOS antibody is vopratelimab. In certain preferred embodiments, the anti-ICOS antibody is an antibody having light and heavy chain sequences corresponding to SEQ ID NOs: 30 and 31, respectively.

b. Anti-CTLA4 Antibodies

In some embodiments, an anti-ICOS antibody is administered in combination with an anti-CTLA4 antibody, such as an anti-CTLA4 antagonist antibody. An anti-CTLA-4 antagonist antibody refers to an agent capable of inhibiting the activity of cytotoxic T-lymphocyte-associated protein 4 (CTLA4), thereby activating the immune system. The CTLA-4 antibody may bind to CTLA4 and reverse CTLA-4-mediated immunosuppression. A non-limiting exemplary anti-CTLA4 antibody is ipilimumab (YERVOY®, BMS), which may be administered according to methods known in the art, e.g., as approved by the US FDA. For example, ipilimumab may be administered in the amount of 3 mg/kg intravenously over 90 minutes every three weeks for a total of 4 doses (unresectable or metastatic melanoma); or at 10 mg/kg intravenously over 90 minutes every three weeks for a total of 4 doses, followed by 10 mg/kg every 12 weeks for up to 3 years or until documented recurrence or unacceptable toxicity (adjuvant melanoma).

In some embodiments, the anti-CTLA4 antibody used in the methods provided herein is ipilimumab. In some embodiments, each dose of the anti-CTLA4 antagonist antibody is 3 mg/kg. In some such embodiments, the anti-CTLA4 antibody is administered every six weeks.

Further non-limiting exemplary anti-CTLA4 antibodies include tremelimumab; AGEN1181 (Agenus); AGEN1884 (Agenus); AGEN2041 (Agenus); and IBI310 (Innovent Biologics). In some embodiments, a method comprises administering an anti-ICOS antibody in combination with tremelimumab according to a treatment schedule provided herein.

c. OX40 Antibodies

In some embodiments, the additional anti-cancer therapy is an anti-OX40 agonist antibody. An OX40 agonist antibody refers to an agent that induces the activity of OX40, thereby activating the immune system and enhancing anti-tumor activity. Nonlimiting, exemplary agonist anti-OX40 antibodies include Medi6469 (MedImmune) and MOXR0916/RG7888 (Roche). These antibodies may be administered according to methods and in regimens determined to be appropriate by those of skill in the art.

d. TIGIT Antibodies

In some embodiments, the additional anti-cancer therapy is an anti-TIGIT antibody that is capable of antagonizing or inhibiting the activity of T-cell immunoreceptor with Ig and ITIM domains (TIGIT), thereby reversing TIGIT-mediated immunosuppression. Non-limiting exemplary TIGIT antibodies include OMP-313M32, BMS-986207, and the antibodies disclosed in PCT Publication Nos. WO2016028656 and WO2017053748, and U.S. Publication Nos. US20170281764 and US20160376365. These agents may be administered according to methods and in regimens determined to be appropriate by those of skill in the art.

e. IDO Inhibitors

In some embodiments, the additional anti-cancer therapy is an IDO inhibitor. An IDO inhibitor refers to an agent capable of inhibiting the activity of indoleamine 2,3-dioxygenase (IDO) and thereby reversing IDO-mediated immunosuppression. The IDO inhibitor may inhibit IDOL and/or ID02 (INDOL1). An IDO inhibitor may be a reversible or irreversible IDO inhibitor. A reversible IDO inhibitor is a compound that reversibly inhibits IDO enzyme activity either at the catalytic site or at a non-catalytic site while an irreversible IDO inhibitor is a compound that irreversibly inhibits IDO enzyme activity by forming a covalent bond with the enzyme. Non-limiting exemplary IDO inhibitors are described, e.g., in US 2016/0060237; and US 2015/0352206. Nonlimiting exemplary IDO inhibitors include indoximod (New Link Genetics), INCB024360 (Incyte Corp), 1-methyl-D-tryptophan (New Link Genetics), and GDC-0919 (Genentech/New Link Genetics). These agents may be administered according to methods and in regimens determined to be appropriate by those of skill in the art.

f. RORγ Agonists

In some embodiments, the additional anti-cancer therapy is a RORγ agonist. RORγ agonists refer to an agent capable of inducing the activity of retinoic acid-related orphan receptor gamma (RORγ), thereby decreasing immunosuppressive mechanisms. Non-limiting exemplary RORγ agonists include, but are not limited to, LYC-55716 (Lycera/Celgene) and INV-71 (Innovimmune). These agents may be administered according to methods and in regimens determined to be appropriate by those of skill in the art.

g. Chemotherapies

In some embodiments, an additional therapeutic agent is a chemotherapeutic agent. Exemplary chemotherapeutic agents that may be combined with the anti-ICOS antibodies provided herein include, but are not limited to, capecitabine, cyclophosphamide, dacarbazine, temozolomide, cyclophosphamide, docetaxel, doxorubicin, daunorubicin, cisplatin, carboplatin, epirubicin, eribulin, 5-FU, gemcitabine, irinotecan, ixabepilone, methotrexate, mitoxantrone, oxaliplatin, paclitaxel, nab-paclitaxel, ABRAXANE® (protein-bound paclitaxel), pemetrexed, vinorelbine, and vincristine. In some embodiments, an anti-ICOS antibody provided herein is administered with at least one kinase inhibitor. Nonlimiting exemplary kinase inhibitors include erlotinib, afatinib, gefitinib, crizotinib, dabrafenib, trametinib, vemurafenib, and cobimetinib. These agents may be administered according to methods and in regimens determined to be appropriate by those of skill in the art.

h. Cancer Vaccines

In some embodiments, an additional therapeutic agent is a cancer vaccine. Cancer vaccines have been investigated as a potential approach for antigen transfer and activation of dendritic cells. In particular, vaccination in combination with immunologic checkpoints or agonists for co-stimulatory pathways have shown evidence of overcoming tolerance and generating increased anti-tumor response. A range of cancer vaccines have been tested that employ different approaches to promoting an immune response against the tumor (see, e.g., Emens L A, Expert Opin Emerg Drugs 13(2): 295-308 (2008)). Approaches have been designed to enhance the response of B cells, T cells, or professional antigen-presenting cells against tumors. Exemplary types of cancer vaccines include, but are not limited to, peptide-based vaccines that employ targeting distinct tumor antigens, which may be delivered as peptides/proteins or as genetically-engineered DNA vectors, viruses, bacteria, or the like; and cell biology approaches, for example, for cancer vaccine development against less well-defined targets, including, but not limited to, vaccines developed from patient-derived dendritic cells, autologous tumor cells or tumor cell lysates, allogeneic tumor cells, and the like.

Exemplary cancer vaccines include, but are not limited to, dendritic cell vaccines, oncolytic viruses, tumor cell vaccines, etc. In some embodiments, such vaccines augment the anti-tumor response. Examples of cancer vaccines that can be used in combination with anti-ICOS antibodies provided herein include, but are not limited to, MAGE3 vaccine (e.g., for melanoma and bladder cancer), MUC1 vaccine (e.g., for breast cancer), EGFRv3 (such as Rindopepimut, e.g., for brain cancer, including glioblastoma multiforme), or ALVAC-CEA (e.g., for CEA+ cancers).

Nonlimiting exemplary cancer vaccines also include Sipuleucel-T, which is derived from autologous peripheral-blood mononuclear cells (PBMCs) that include antigen-presenting cells (see, e.g., Kantoff P W et al., N Engl J Med 363:411-22 (2010)). In Sipuleucel-T generation, the patient's PBMCs are activated ex vivo with PA2024, a recombinant fusion protein of prostatic acid phosphatase (a prostate antigen) and granulocyte-macrophage colony-stimulating factor (an immune-cell activator). Another approach to a candidate cancer vaccine is to generate an immune response against specific peptides mutated in tumor tissue, such as melanoma (see, e.g., Carreno B M et al., Science 348:6236 (2015)). Such mutated peptides may, in some embodiments, be referred to as neoantigens. As a nonlimiting example of the use of neoantigens in tumor vaccines, neoantigens in the tumor predicted to bind the major histocompatibility complex protein HLA-A*02:01 are identified for individual patients with a cancer, such as melanoma. Dendritic cells from the patient are matured ex vivo, then incubated with neoantigens. The activated dendritic cells are then administered to the patient. In some embodiments, following administration of the cancer vaccine, robust T-cell immunity against the neoantigen is detectable.

In some such embodiments, the cancer vaccine is developed using a neoantigen. In some embodiments, the cancer vaccine is a DNA vaccine. In some embodiments, the cancer vaccine is an engineered virus comprising a cancer antigen, such as PROSTVAC (rilimogene galvacirepvec/rilimogene glafolivec). In some embodiments, the cancer vaccine comprises engineered tumor cells, such as GVAX, which is a granulocyte-macrophage colony-stimulating factor (GM-CSF) gene-transfected tumor cell vaccine (see, e.g., Nemunaitis, 2005, Expert Rev Vaccines, 4: 259-74).

The vaccines may be administered according to methods and in regimens determined to be appropriate by those of skill in the art.

i. Additional Exemplary Anti-Cancer Therapies

Further non-limiting, exemplary anti-cancer therapies include Luspatercept (Acceleron Pharma/Celgene); Motolimod (Array BioPharma/Celgene/VentiRx Pharmaceuticals/Ligand); GI-6301 (GlobeImmune/Celgene/NantWorks); GI-6200 (Globelmmune/Celgene/NantWorks); BLZ-945 (Celgene/Novartis); and ARRY-382 (Array BioPharma/Celgene). These and other agents may be administered according to methods and in regimens determined to be appropriate by those of skill in the art. In some embodiments, the one or more anti-cancer therapies includes surgery and/or radiation therapy. Accordingly, the anti-cancer therapies can optionally be utilized in the adjuvant or neoadjuvant setting.

In some embodiments, the additional therapeutic agent is an immune-modifying drug (IMiD). Nonlimiting exemplary IMiDs include thalidomide, lenalidomide, and pomalidomide.

In some embodiments, the additional anti-cancer therapy is a therapeutic antibody selected from cetuximab (such as ERBITUX®), elotuzumab (such as EMPLICITI®), rituximab (such as RITUXIN®), trastuzumab (such as HERCEPTIN®), and atezolizumab (such as TECENTRIQ®).

In some embodiments, the additional anti-cancer therapy is a chimeric antigen receptor T cell therapy (CAR-T therapy).

In some embodiments, the additional anti-cancer therapy is a Vascular Endothelial Growth Factor (VEGF) receptor inhibitor, such as, but not limited to, bevacizumab (Avastin®), axitinib (Inlyta®); brivanib alaninate (BMS-582664, (S)—((R)-1-(4-(4-Fluoro-2-methyl-1H-indol-5-yloxy)-5-methylpyrrolo[2,1-f][1,2,4]triazin-6-yloxy)propan-2-yl)2-aminopropanoate); sorafenib (Nexavar®); pazopanib (Votrient®); sunitinib malate (Sutent®); cediranib (AZD2171, CAS 288383-20-1); vargatef (BIBF1120, CAS 928326-83-4); foretinib (GSK1363089); telatinib (BAY57-9352, CAS 332012-40-5); apatinib (YN968D1, CAS 811803-05-1); imatinib (Gleevec®); ponatinib (AP24534, CAS 943319-70-8); tivozanib (AV951, CAS 475108-18-0); regorafenib (BAY73-4506, CAS 755037-03-7); vatalanib dihydrochloride (PTK787, CAS 212141-51-0); brivanib (BMS-540215, CAS 649735-46-6); vandetanib (Caprelsa® or AZD6474); motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); dovitinib dilactic acid (TKI258, CAS 852433-84-2); linfanib (ABT869, CAS 796967-16-3); cabozantinib (XL184, CAS 849217-68-1); lestaurtinib (CAS 111358-88-4); N-[5-[[[5-(1,1-Dimethylethyl)-2-oxazolyl]methyl]thio]-2-thiazolyl]-4-piperidinecarboxamide (BMS38703, CAS 345627-80-7); (3R,4R)-4-Amino-1-((4-((3-methoxyphenyl)amino)pyrrolo[2,1-f][1,2,4]triazin-5-yl)methyl)piperidin-3-ol (BMS690514); N-(3,4-Dichloro-2-fluorophenyl)-6-methoxy-7-[[(3aα,5β, 6aα)-octahydro-2-methylcyclopenta[c]pyrrol-5-yl]methoxy]-4-quinazolinamine (XL647, CAS 781613-23-8); 4-Methyl-3-[[1-methyl-6β-pyridinyl)-1H-pyrazolo[3,4-d]pyrimidin-4-yl]amino]-N-[3-(trifluoromethyl)phenyl]-benzamide (BHG712, CAS 940310-85-0); and aflibercept (Eylea®).

In some embodiments, the additional anti-cancer therapy is a cytokine therapy, such as in combination with one, two, three or more cytokines. In some embodiments, the cytokine is one, two, three or more interleukins (ILs) chosen from IL-1, IL-2, IL-12, IL-15 or IL-21.

In some embodiments, the additional anti-cancer therapy is a cytokine therapy in combination with an agent that targets PTEN. Without intending to be bound by any particular theory, it is believed that enhanced PI3K signaling reduces Treg function.

In some embodiments, the additional anti-cancer therapy is an A2A receptor antagonist. In some embodiments, the A2aR antagonist is an A2aR pathway antagonist (e.g., a CD-73 inhibitor, such as an anti-CD73 antibody). A nonlimiting exemplary anti-CD73 antibody is MEDI9447. Without intending to be bound by any particular theory, targeting the extracellular production of adenosine by CD73 may reduce the immunosuppressive effects of adenosine. MEDI9447 has been reported to have a range of activities, including, for example, inhibition of CD73 ectonucleotidase activity, relief from AMP-mediated lymphocyte suppression, and inhibition of syngeneic tumor growth. In some embodiments, an anti-ICOS antibody provided herein is administered in combination with one or more of the following: i) an agonist of Stimulator of Interferon Genes (a STING agonist), (ii) an agonist of a Toll-Like Receptor (TLR) (such as an agonist of TLR-3, -4, -5, -7, -8, or -9), (iii) a TIM-3 modulator (such as an anti-TIM-3 antibody), (iv) a VEGF receptor inhibitor, (v) a c-Met inhibitor, (vi) a TGFβ inhibitor (such as an anti-TGFβ antibody), (vii) an A2AR antagonist, and/or a (viii) BTK inhibitor.

In some embodiments, an oncolytic virus is a recombinant oncolytic virus, such as those described in US2010/0178684 A1, which is incorporated herein by reference in its entirety. In some embodiments, a recombinant oncolytic virus comprises a nucleic acid sequence (e.g., heterologous nucleic acid sequence) encoding an inhibitor of an immune or inflammatory response, e.g., as described in US2010/0178684 A1. In some embodiments, a recombinant oncolytic virus, such as oncolytic NDV, comprises a nucleic acid sequence encoding a pro-apoptotic protein (such as apoptin), a cytokine (such as GM-CSF, CSF, interferon-gamma, interleukin-2 (IL-2), or tumor necrosis factor-alpha), an immunoglobulin (such as an antibody against ED-B firbonectin), a tumor associated antigen, a bispecific adapter protein (such as a bispecific antibody or antibody fragment directed against NDV HN protein and a T cell co-stimulatory receptor, such as CD3 or CD28; or a fusion protein between human IL-2 and a single chain antibody directed against NDV HN protein). See, e.g., Zamarin et al. Future Microbiol. 7.3(2012):347-67, which is incorporated herein by reference in its entirety. In some embodiments, the oncolytic virus is a chimeric oncolytic NDV, e.g., as described in U.S. Pat. No. 8,591,881 B2, US 2012/0122185 A1, and/or US 2014/0271677 A1, each of which is incorporated herein by reference in its entirety.

In some embodiments, an oncolytic virus comprises a conditionally replicative adenovirus (CRAd), which is designed to replicate exclusively in cancer cells. See, e.g., Alemany et al. Nature Biotechnol. 18(2000):723-27, which is incorporated herein by reference in its entirety. In some embodiments, an oncolytic adenovirus comprises one described in Table 1 on page 725 of Alemany et al..

Exemplary oncolytic viruses include but are not limited to the following:
Group B Oncolytic Adenovirus (ColoAdl) (PsiOxus Therapeutics Ltd.) (see, e.g., Clinical Trial Identifier: NCT02053220);
ONCOS-102 (previously called CGTG-102), which is an adenovirus comprising granulocyte-macrophage colony stimulating factor (GM-CSF) (Oncos Therapeutics) (see, e.g., Clinical Trial Identifier: NCT01598129);
VCN-01, which is a genetically modified oncolytic human adenovirus encoding human PH20 hyaluronidase (VCN Biosciences, S.L.) (see, e.g., Clinical Trial Identifiers: NCT02045602 and NCT02045589);
Conditionally Replicative Adenovirus ICOVIR-5, which is a virus derived from wild-type human adenovirus serotype 5 (Had5) that has been modified to selectively replicate in cancer cells with a deregulated retinoblastoma/E2F pathway (Institut Catala d'Oncologia) (see, e.g., Clinical Trial Identifier: NCT01864759);
Celyvir, which comprises bone marrow-derived autologous mesenchymal stem cells (MSCs) infected with ICOVIR5, an oncolytic adenovirus (Hospital Infantil Universitario Nino Jesus, Madrid, Spain/Ramon Alemany) (see, e.g., Clinical Trial Identifier: NCT01844661); and
CG0070, which is a conditionally replicating oncolytic serotype 5 adenovirus (Ad5) in which human E2F-1 promoter drives expression of the essential Ela viral genes, thereby restricting viral replication and cytotoxicity to Rb pathway-defective tumor cells (Cold Genesys, Inc.) (see, e.g., Clinical Trial Identifier: NCT02143804); or DNX-2401 (formerly named Delta-24-RGD), which is an adenovirus that has been engineered to replicate selectively in retinoblastoma (Rb)-pathway deficient cells and to infect cells that express certain RGD-binding integrins more efficiently (Clinica Universidad de Navarra, Universidad de Navarra/DNAtrix, Inc.) (see, e.g., Clinical Trial Identifier: NCT01956734).

Exemplary BTK inhibitors include, but are not limited to, ibrutinib (PCI-32765); GDC-0834; RN-486; CGI-560; CGI-1764; HM-71224; CC-292; ONO-4059; CNX-774; or LFM-A13. In some embodiments, a BTK inhibitor does not reduce or inhibit the kinase activity of interleukin-2-inducible kinase (ITK). In some such embodiments, the BTK inhibitor is selected from GDC-0834; RN-486; CGI-560; CGI-1764; HM-71224; CC-292; ONO-4059; CNX-774; or LFM-A13. In some embodiments, a kinase inhibitor is a BTK inhibitor, such as ibrutinib (PCI-32765).

In some embodiments, the additional anti-cancer therapy is an IL-33 and/or IL-33R inhibitors (such as, for example, an anti-IL-33 antibody or an anti-IL-33R antibody).

In some embodiments, the additional anti-cancer therapy is an acyl coenzyme A-cholesterol acyltransferase (ACAT) inhibitor, such as avasimibe (CI-1011).

In some embodiments, the additional anti-cancer therapy is an inhibitor of chemokine (C-X-C motif) receptor 2 (CXCR2). In some embodiments, the CXCR2 inhibitor is danirixin (CAS Registry Number: 954126-98-8). Danirixin is also known as GSK1325756 or 1-(4-chloro-2-hydroxy-3-piperidin-3-ylsulfonylphenyl)-3-(3-fluoro-2-methylphenyl)urea, and is described, e.g., in Miller et al. Eur J Drug Metab Pharmacokinet (2014) 39:173-181; and Miller et al. BMC Pharmacology and Toxicology (2015), 16:18. In some embodiments, the CXCR2 inhibitor is reparixin (CAS Registry Number: 266359-83-5). Reparixin is also known as repertaxin or (2R)-2-[4-(2-methylpropyl)phenyl]-N-methylsulfonylpropanamide, and is a non-competitive allosteric inhibitor of CXCR1/2. Reparixin is described, e.g., in Zarbock et al. British Journal of Pharmacology (2008), 1-8. In some embodiments, the CXCR2 inhibitor is navarixin. Navarixin is also known as MK-7123, SCH 527123, PS291822, or 2-hydroxy-N,N-dimethyl-3-[[2-[[(1R)-1-(5-methylfuran-2-yl)propyl]amino]-3,4-dioxocyclobuten-1-yl]amino]benzamide, and is described, e.g., in Ning et al. Mol Cancer Ther. 2012; 11(6):1353-64.

In some embodiments, the additional anti-cancer therapy is a CD27 agonist. In some embodiments, the CD27 agonist is varlilumab (CAS Registry Number: 1393344-72-3). Varlilumab is also known as CDX-1127 (Celldex) or 1F5, and is a fully human monoclonal antibody that targets CD27. Varlilumab activates human T cells in the context of T cell receptor stimulation and therefore mediates anti-tumor effects. Varlilumab also provides direct therapeutic effects against tumors that express CD27. Varlilumab is described, e.g., in Vitale et al., Clin Cancer Res. 2012; 18(14):3812-21, WO 2008/051424, and U.S. Pat. No. 8,481,029. In some embodiments, the CD27 agonist is BION-1402 (BioNovion), which is also known as hCD27.15. BION-1402 is an anti-human CD27 monoclonal antibody that stimulates the proliferation and/or survival of CD27+ cells. BION-1402 activates human CD27 more effectively than its ligand CD70, which results in a significantly increased effect on proliferation of CD8+ and CD4+ T-cells. BION-1402 is disclosed, e.g., as hCD27.15 in WO 2012/004367. The antibody is produced by hybridoma hCD27.15, which was deposited with the ATCC in on Jun. 2, 2010 under number PTA-11008.

### EXAMPLES

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1: Pharmacokinetic Study

A pharmacokinetic study of JTX-4014 in patients was carried out as follows. Q3W (one dose every three weeks) dosing of JTX-4014 at 80, 240, 400, 800, and 1200 mg/kg, as well as Q6W (once every six weeks) dosing at 800 mg/kg was conducted. Three patients per group received a single dose of JTX-4014 at the indicated dose by intravenous infusion. Blood samples were collected at 0, 1, 3, 7, 14, and 21 days for the Q3W groups, and also at 42 days for the Q6W group, and the serum concentration of JTX-4014 was determined by Meso Scale Discovery (MSD) system. A MSD Multi-Array plate was coated with monoclonal antibody against JTX-4014 at a concentration of 0.5 μg/mL overnight. Samples containing JTX-4014 were incubated on the coated plate for 60 mins at room temperature. The bound JTX-4014 was detected with 0.125 μg/mL of biotinylated mouse anti-human antibody for 60 mins, followed by addition of streptavidin-ruthenium at 0.125 μg/mL for 30 mins. Upon application of an electrical charge, an ECL signal was produced and detected with MSD instrument. The JTX-4014 concentration was quantified based on ECL signal. The JTX-4014 Concentration time course was analyzed by non-compartment analysis in Phoenix 8.0.

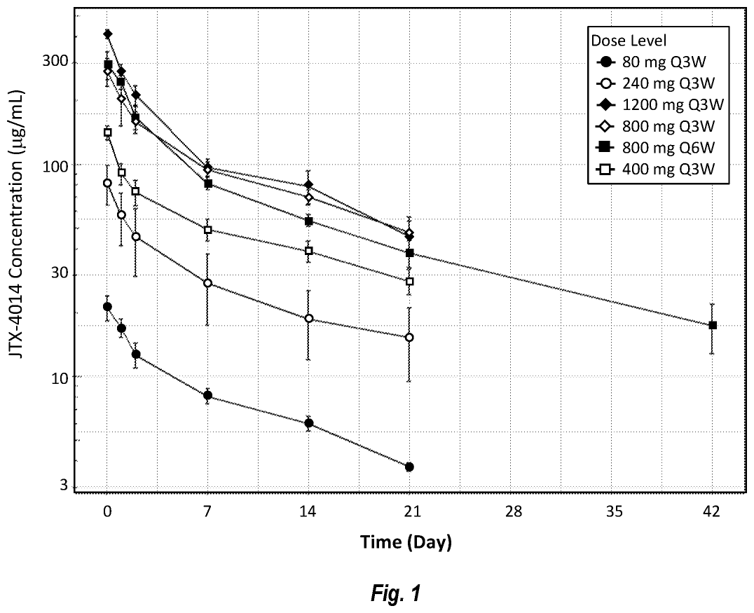

As shown in FIG. 1, moderate PK variability was observed, with minimal to moderate PK accumulation. An approximately dose-proportional increase in exposure was observed, with a terminal half-life of about 11-17 days.

### Example 2: Pharmacokinetic Simulations

Serum concentration-time profiles of JTX-4014 at different clinical dose regimens were analyzed on the basis of a population pharmacokinetic model. The model was developed on the data described in Example 1, from the 6 cohorts (18 subjects). The model was structured as a two-compartment model with linear elimination from the central compartment, and was parameterized using clearance and volume terms. To account for variability in JTX-4014 pharmacokinetics between subjects, interindividual variability was estimated on the clearance from the central compartment and both volume terms associated with the central and peripheral compartment. The variability terms were implemented as exponential random effect models at the subject level, corresponding to log-normally distributed individual parameters. Residual variability in JTX-4014 serum concentrations was described by a proportional random effects model at the observation level. The appropriateness of the model to describe the JTX-4014 concentration-time data was established through a variety of goodness-of-fit plots as well as a visual predictive check comparing simulated data from the model to the actual observed data.

Using the established model, JTX-4014 serum concentration-time profiles were predicted/simulated for a range of JTX-4014 multiple dose regimens, encompassing a range of dose levels (80 to 1200 mg) as well as different dosing frequencies (every 2, 3, 4 or 6 weeks). For each dosing regimen, concentration-time profiles were predicted for 200 subjects, by sampling from the interindividual and residual random effect distributions, and subsequently summarized per dosing regimen as median prediction and 95% prediction interval. Model development as well as the simulations were performed using NONMEM 7.4 (ICON Development Solutions, Hanover, Md.).

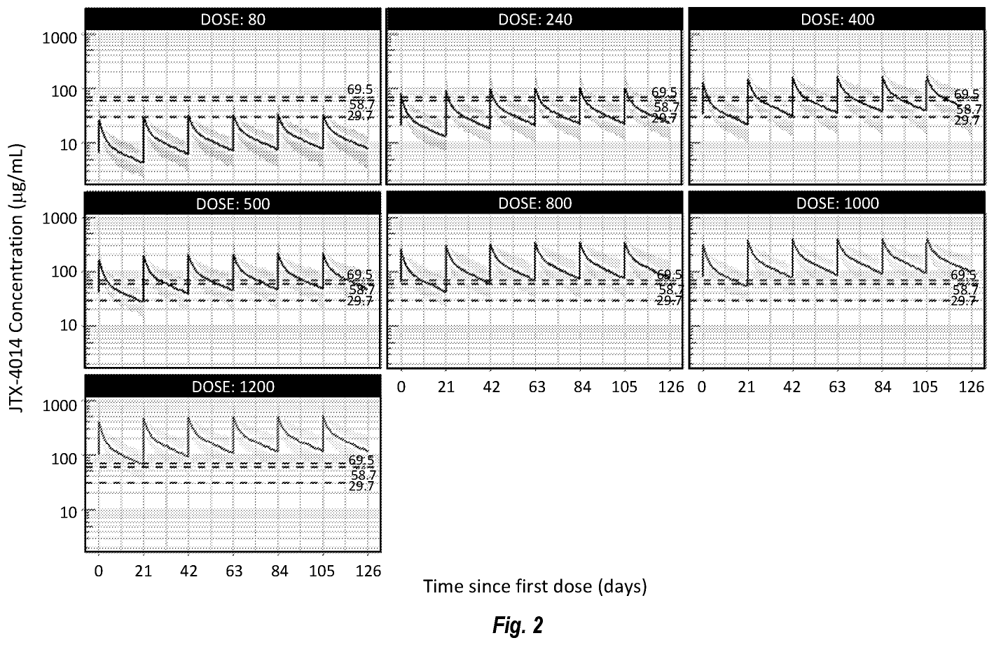

FIG. 2 shows simulated serum concentration over time for Q3W dosing at 80, 240, 400, 500, 800, 1000, and 1200 mg/kg for 6 doses for a period of 18 weeks.

As shown in Table 2, steady-state trough serum concentrations (C_{trough, ss}) were comparable to the reported mean/median C_{trough, ss} of approved anti-PD-1 antibodies: Opdivo (240 mg Q2W): 69.5 μg/mL; Libtayo (350 mg Q3W): 58.7 μg/mL; Keytruda (200 mg Q3W): 29.7 μg/mL. See, e.g., Freshwater et al., J. Immunother. Canc. 5:43 (2017); Long et al., Annals Oncology 29: 2208-2213 (2018); BLA 761097 for cemiplimab-RWLC (Libtayo). C_{trough, ss} with dosing at 400 mg/kg of JTX-4014 or higher was similar or higher than C_{trough, ss} of Keytruda (200 mg Q3W).

TABLE 2

| Dose | Ctrough, ss (μg/mL) |
|---|---|
| (mg) | median (95% PI) |
| 80 | 7.68 |
| | ( 3.69, 12.98 ) |
| 240 | 22.63 |
| | ( 10.31, 46.45 ) |
| 400 | 38.62 |
| | ( 14.30, 76.21 ) |
| 500 | 47.46 |
| | ( 22.12, 96.51 ) |
| 800 | 72.43 |
| | ( 35.41 , 157.22 ) |
| 1000 | 97.72 |
| | ( 38.95 , 183.25 ) |
| 1200 | 114.66 |
| | ( 51.20, 217.14 ) |

As shown in Table 3, steady-state trough serum concentrations (C_{trough, ss}) were comparable to the reported mean/median C_{trough, ss} of approved anti-PD-1 antibodies: Opdivo (240 mg Q2W), Libtayo (350 mg Q3W); Keytruda (200 mg Q3W), noted above. C_{trough, ss} with dosing at 1000 mg/kg of JTX-4014 or higher was similar or higher than C_{trough, ss} of Keytruda (200 mg Q3W).

TABLE 3

| Dose | Ctrough, ss (μg/mL) |
|---|---|
| (mg) | median (95% PI) |
| 800 | 23.07 |
| | ( 7.94, 54.69 ) |
| 1000 | 29.70 |
| | ( 8.77, 71.24 ) |
| 1200 | 32.76 |
| | ( 11.34, 84.17 ) |

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

Table of Sequences

| SEQ | | |
|---|---|---|
| ID NO | Description | Sequence |
| 1 | Human PD-1 | MQIPQAPWPV VWAVLQLGWR PGWFLDSPDR PWNPPTFSPA LLVVTEGDNA |
| | amino acid | TFTCSFSNTS ESFVLNWYRM SPSNQTDKLA AFPEDRSQPG QDCRFRVTQL |
| | sequence; | PNGRDFHMSV VRARRNDSGT YLCGAISLAP KAQIKESLRA ELRVTERRAE |
| | UniProtKB/ | VPTAHPSPSP RPAGQFQTLV VGVVGGLLGS LVLLVWVLAV ICSRAARGTI |
| | Swiss-Prot: | GARRTGQPLK EDPSAVPVFS VDYGELDFQW REKTPEPPVP CVPEQTEYAT |
| | Q15116; | IVFPSGMGTS SPARRGSADG PRSAQPLRPE DGHCSWPL |
| | 1 Aug. 2016 | |
| 4 | Mature human | PGWFLDSPDR PWNPPTFSPA LLVVTEGDNA TFTCSFSNTS ESFVLNWYRM |
| | PD-1 amino | SPSNQTDKLA AFPEDRSQPG QDCRFRVTQL PNGRDFHMSV VRARRNDSGT |
| | acid sequence | YLCGAISLAP KAQIKESLRA ELRVTERRAE VPTAHPSPSP RPAGQFQTLV |
| | (without signal | VGVVGGLLGS LVLLVWVLAV ICSRAARGTI GARRTGQPLK EDPSAVPVFS |
| | sequence) | VDYGELDFQW REKTPEPPVP CVPEQTEYAT IVFPSGMGTS SPARRGSADG |
| | | PRSAQPLRPE DGHCSWPL |
| 2 | Mouse PD-1; | MWVRQVPWSF TWAVLQLSWQ SGWLLEVPNG PWRSLTFYPA WLTVSEGANA |
| | UniProtKB/ | TFTCSLSNWS EDLMLNWNRL SPSNQTEKQA AFCNGLSQPV QDARFQIIQL |
| | Swiss-Prot: | PNRHDFHMNI LDTRRNDSGI YLCGAISLHP KAKIEESPGA ELVVTERILE |
| | Q02242; | TSTRYPSPSP KPEGRFQGMV IGIMSALVGI PVLLLLAWAL AVFCSTSMSE |
| | 1 Aug. 2016 | ARGAGSKDDT LKEEPSAAPV PSVAYEELDF QGREKTPELP TACVHTEYAT |
| | | IVFTEGLGAS AMGRRGSADG LQGPRPPRHE DGHCSWPL |
| 5 | Mature mouse | SGWLLEVPNG PWRSLTFYPA WLTVSEGANA TFTCSLSNWS EDLMLNWNRL |
| | PD-1 amino | SPSNQTEKQA AFCNGLSQPV QDARFQIIQL PNRHDFHMNI LDTRRNDSGI |
| | acid sequence | YLCGAISLHP KAKIEESPGA ELVVTERILE TSTRYPSPSP KPEGRFQGMV |
| | (without signal | IGIMSALVGI PVLLLLAWAL AVFCSTSMSE ARGAGSKDDT LKEEPSAAPV |
| | sequence) | PSVAYEELDF QGREKTPELP TACVHTEYAT IVFTEGLGAS AMGRRGSADG |
| | | LQGPRPPRHE DGHCSWPL |
| 3 | Cynomolgus | MQIPQAPWPV VWAVLQLGWR PGWFLDSPDR PWNPPTFSPA LLVVTEGDNA |
| | Monkey PD-1; | TFTCSFSNTS ESFVLNWYRM SPSNQTDKLA AFPEDRSQPG QDCRFRVTQL |
| | UniProtKB/ | PNGRDFHMSV VRARRNDSGT YLCGAISLAP KAQIKESLRA ELRVTERRAE |
| | Swiss-Prot: | VPTAHPSPSP RPAGQFQTLV VGVVGGLLGS LVLLVWVLAV ICSRAARGTI |
| | B0LAJ3; | GARRTGQPLK EDPSAVPVFS VDYGELDFQW REKTPEPPVP CVPEQTEYAT |
| | 1 Aug. 2016 | IVFPSGMGTS SPARRGSADG PRSAQPLRPE DGHCSWPL |
| 6 | Mature | PGWFLDSPDR PWNPPTFSPA LLVVTEGDNA TFTCSFSNTS ESFVLNWYRM |
| | cynomolgus | SPSNQTDKLA AFPEDRSQPG QDCRFRVTQL PNGRDFHMSV VRARRNDSGT |
| | monkey PD-1 | YLCGAISLAP KAQIKESLRA ELRVTERRAE VPTAHPSPSP RPAGQFQTLV |
| | (without signal | VGVVGGLLGS LVLLVWVLAV ICSRAARGTI GARRTGQPLK EDPSAVPVFS |
| | sequence) | VDYGELDFQW REKTPEPPVP CVPEQTEYAT IVFPSGMGTS SPARRGSADG |
| | | PRSAQPLRPE DGHCSWPL |
| 20 | JTX-4014 | QVQLVQSGAEVKKPGASVKVSCKASGYTFPSYYMHWVRQAPGQGL |
| | VH Sequence | EWMGIINPEGGSTAYAQKFQGRVTMTRDTSTSTVYMELSSLRSEDT |
| | | AVYYCARGGTYYDYTYWGQGTLVTVSS |
| 21 | JTX-4014 | YTFPSYYMH |
| | HCDR1 | |
| 22 | JTX-4014 | IINPEGGSTAYAQKFQG |
| | HCDR2 | |
| 23 | JTX-4014 | ARGGTYYDYTY |
| | HCDR3 | |
| 24 | JTX-4014 | DIQMTQSPSTLSASVGDRVTITCRASQSISSWLAWYQQKP |
| | VL Sequence | GKAPKLLIYEASSLESGVPSRFSGSGSGTEFTLTISSLQPDDF |
| | | ATYYCQQYNSFPPTFGGGTKVEIK |
| 25 | JTX-4014 | RASQSISSWLA |
| | LCDR1 | |
| 26 | JTX-4014 | EASSLES |
| | LCDR2 | |
| 27 | JTX-4014 | QQYNSFPPT |
| | LCDR3 | |
| 28 | JTX-4014 | QVQLVQSGAE VKKPGASVKV SCKASGYTFP SYYMHWVRQA PGQGLEWMGI |
| | heavy chain | INPEGGSTAY AQKFQGRVTM TRDTSTSTVY MELSSLRSED TAVYYCARGG |
| | | TYYDYTYWGQ GTLVTVSSAS TKGPSVFPLA PCSRSTSEST AALGCLVKDY |
| | | FPEPVTVSWN SGALTSGVHT FPAVLQSSGL YSLSSVVTVP SSSLGTKTYT |
| | | CNVDHKPSNT KVDKRVESKY GPPCPPCPAP EFLGGPSVFL FPPKPKDTLM |
| | | ISRTPEVTCV VVDVSQEDPE VQFNWYVDGV EVHNAKTKPR EEQFNSTYRV |
| | | VSVLTVLHQD WLNGKEYKCK VSNKGLPSSI EKTISKAKGQ PREPQVYTLP |
| | | PSQEEMTKNQ VSLTCLVKGF YPSDIAVEWE SNGQPENNYK TTPPVLDSDG |
| | | SFFLYSRLTV DKSRWQEGNV FSCSVMHEAL HNHYTQKSLS LSLG |
| 29 | JTX-4014 light | DIQMTQSPST LSASVGDRVT ITCRASQSIS SWLAWYQQKP GKAPKLLIYE |
| | chain | ASSLESGVPS RFSGSGSGTE FTLTISSLQP DDFATYYCQQ YNSFPPTFGG |
| | | GTKVEIKRTV AAPSVFIFPP SDEQLKSGTA SVVCLLNNFY PREAKVQWKV |
| | | DNALQSGNSQ ESVTEQDSKD STYSLSSTLT LSKADYEKHK VYACEVTHQG |
| | | LSSPVTKSFN RGEC |
| 30 | vopratelimab | EVQLVESGGG LVQPGGSLRL SCAASGFTFS DYWMDWVRQA PGKGLVWVSN |
| | heavy chain | IDEDGSITEY SPFVKGRFTI SRDNAKNTLY LQMNSLRAED TAVYYCTRWG |
| | | RFGFDSWGQG TLVTVSSAST KGPSVFPLAP SSKSTSGGTA ALGCLVKDYF |
| | | PEPVTVSWNS GALTSGVHTF PAVLQSSGLY SLSSVVTVPS SSLGTQTYIC |
| | | NVNHKPSNTK VDKKVEPKSC DKTHTCPPCP APELLGGPSV FLFPPKPKDT |
| | | LMISRTPEVT CVVVDVSHED PEVKFNWYVD GVEVHNAKTK PREEQYNSTY |
| | | RVVSVLTVLH QDWLNGKEYK CKVSNKALPA PIEKTISKAK GQPREPQVYT |
| | | LPPSREEMTK NQVSLTCLVK GFYPSDIAVE WESNGQPENN YKTTPPVLDS |
| | | DGSFFLYSKL TVDKSRWQQG NVFSCSVMHE ALHNHYTQKS LSLSPG |
| 31 | vopretelimab | DIVMTQSPDS LAVSLGERAT INCKSSQSLL SGSFNYLTWY QQKPGQPPKL |
| | light chain | LIFYASTRHT GVPDRFSGSG SGTDFTLTIS SLQAEDVAVY YCHHHYNAPP |
| | | TFGPGTKVDI KRTVAAPSVF IFPPSDEQLK SGTASVVCLL NNFYPREAKV |
| | | QWKVDNALQS GNSQESVTEQ DSKDSTYSLS STLTLSKADY EKHKVYACEV |
| | | THQGLSSPVT KSFNRGEC |

What is claimed is:

1. A method of treating cancer in a subject, comprising administering a dose of an anti-PD-1 antibody to said subject either at 1000 mg/kg once every 6 weeks or at 400-600 mg/kg once every 3 weeks; wherein the anti-PD-1 antibody comprises a heavy chain complementarity determining region 1 (HCDR1) comprising the amino acid sequence of SEQ ID NO: 21, a HCDR2 comprising the amino acid sequence of SEQ ID NO: 22, a HCDR3 comprising the amino acid sequence of SEQ ID NO: 23, a light chain CDR1 (LCDR1) comprising the amino acid sequence of SEQ ID NO: 25, a LCDR2 comprising the amino acid sequence of SEQ ID NO: 26, and a LCDR3 comprising the amino acid sequence of SEQ ID NO: 27.

2. The method of claim 1, wherein the anti-PD-1 antibody comprises a heavy chain variable region comprising the amino acid sequence of SEQ ID NO: 20 and a light chain variable region comprising the amino acid sequence of SEQ ID NO: 24.

3. The method of claim 1, wherein the anti-PD-1 antibody comprises a heavy chain comprising the amino acid sequence of SEQ ID NO: 28 and a light chain comprising the amino acid sequence of SEQ ID NO: 29.

4. The method of any one of claims 1-3, wherein the dose is administered every 6 weeks at 1000 mg/kg.

5. The method of any one of claims 1-3, wherein the dose is administered every 3 weeks at 400-600 mg/kg.

6. The method of claim 5, wherein the dose is administered every 3 weeks at 400 mg/kg.

7. The method of claim 5, wherein the dose is administered every 3 weeks at 500 mg/kg.

8. The method of claim 5, wherein the dose is administered every 3 weeks at 600 mg/kg.

9. The method of any one of claims 1-8, wherein the subject is administered the anti-PD-1 antibody for a period of about 12 weeks.

10. The method of any one of claims 1-8, wherein the subject is administered the anti-PD-1 antibody for a period of about 18 weeks.

11. The method of any one of claims 1-8, wherein the subject is administered the anti-PD-1 antibody for a period of about 24 weeks.

12. The method of any one of claims 1-8, wherein the subject is administered the anti-PD-1 antibody for a period of about 52 weeks.

13. The method of any one of claims 1-12, wherein the subject has a cancer selected from melanoma, non-small cell lung cancer (NSCLC), renal cell carcinoma (RCC) (e.g., clear cell RCC), gastric cancer, bladder cancer, endometrial cancer, MSI-H cancer of any organ, diffuse large B-cell lymphoma (DLBCL), Hodgkin's lymphoma, ovarian cancer (e.g., endometrioid ovarian cancer), head & neck squamous cell cancer (HNSCC), acute myeloid leukemia (AML), rectal cancer, refractory testicular cancer, small cell lung cancer (SCLC), small bowel cancer, metastatic cutaneous squamous cell cancer, cervical cancer, MSI-high colon cancer, esophageal cancer, mesothelioma, breast cancer, and triple negative breast cancer (TNBC).

14. The method of claim 13, wherein the cancer is selected from melanoma, gastric cancer, head & neck squamous cell cancer (HNSCC), non-small cell lung cancer (NSCLC), and triple negative breast cancer (TNBC).

15. The method of any one of claims 1-14, wherein the method comprises administering an anti-PD-1 antibody and at least one additional therapeutic agent.

16. The method of claim 15, wherein the additional therapeutic agent is administered concurrently or sequentially with the anti-PD-1 antibody.

17. The method of claim 15 or claim 16, wherein the additional therapeutic agent is an anti-ICOS antibody.

18. The method of claim 17, wherein the anti-ICOS antibody is GSK3359609, BMS-986226, or KY1044.

19. The method of claim 18, wherein the anti-ICOS antibody is vopratelimab.

20. The method of any one of claims 17-19, wherein each dose of the anti-ICOS agonist antibody is 0.1 mg/kg.

21. The method of any one of claims 17-19, wherein each dose of the anti-ICOS agonist antibody is 0.03 mg/kg.

22. The method of any one of claims 1-21, wherein the subject is a human.
